(19) United States
(12) Patent Application Publication
(10) Pub. No.: US 2026/0053814 A1
(43) Pub. Date: Feb. 26, 2026

(54) TREATMENT REGIMENS FOR GEDATOLISIB IN HORMONALLY-DRIVEN DISORDERS

(71) Applicant: Celcuity Inc., Minneapolis, MN (US)

(72) Inventors: Brian Francis SULLIVAN, Medina, MN (US); Lance Gavin LAING, Orono, MN (US)

(21) Appl. No.: 19/306,599

(22) Filed: Aug. 21, 2025

Related U.S. Application Data
(60) Provisional application No. 63/685,556, filed on Aug. 21, 2024.

(51) Int. Cl. A61K 31/5377; A61K 31/4155; A61K 45/06; A61P 35/00

(52) U.S. Cl. CPC A61K 31/5377; A61K 31/4155; A61K 45/06; A61P 35/00

(57) ABSTRACT

Methods for treating hormonally driven disorders by administering the PI3K/mTOR inhibitor gedatolisib in combination with hormone therapy are provided. Hormonally driven disorders include, for example, prostate cancer and endometrial disorders. The treatment regimens can also include additional therapies, such as chemotherapy, surgery and/or administration of an immune checkpoint inhibitor(s), a receptor tyrosine kinase inhibitor, a CDK 4/6 inhibitor or an epigenetic-targeted therapy.

### RELATED APPLICATION

This application claims priority to U.S. Provisional Application No. 63/685,556 filed Aug. 21, 2024, the entire contents of which is hereby incorporated by reference.

### BACKGROUND

Gedatolisib (PF-05212384; CAS No. 1197160-78-3) is a potent dual inhibitor of PI3K and mTOR that reduces PI3K/AKT/mTOR pathway signaling and induces antitumor activity in cell lines and tumor xenografts models (see e.g., Mallon et al. (2011) Clin. Cancer Res. 17:3193-3203). Mammalian Target of Rapamycin (mTOR) is a cell-signaling protein that regulates the response of tumor cells to nutrient supply and growth factors to control protein translation, cancer cell proliferation, and tumor blood supply through effects on Vascular Endothelial Growth Factor (VEGF). Inhibitors of PI3K and mTOR starve cancer cells and shrink tumors by inhibiting the effect of PI3K and mTOR. Gedatolisib is a competitive inhibitor of the ATP binding site on p110 catalytic subunit of PI3K and mTOR and thus blocks the PI3K and mTOR kinase activity in both of the cellular complexes that have been described for mTOR, mTORC1 and mTORC2. This has at least two important effects. First, mTOR is a downstream mediator of the PI3K/Akt pathway. The PI3K/Akt pathway has been demonstrated to be over activated in numerous cancers and may account for the widespread response from various cancers to mTOR inhibitors. The over-activation of the upstream pathway would normally cause mTOR kinase to be over activated as well. However, in the presence of mTOR inhibitors, this process is blocked. The blocking effect prevents mTOR from signaling to downstream pathways that control protein translation, cell cycling, and cell proliferation and by gedatolisib's effect on PI3K blocks the feedback mechanism of mTOR. Over-activation of the PI3K/Akt kinase pathway is frequently associated with mutations in the p110 catalytic subunit of the PI3K complex and the PTEN pathway repressor gene, that are common in many cancers, constitutively activate the pathway, and may help predict what tumors will respond to PI3K and mTOR inhibitors. Another significant effect of mTOR inhibition is anti-angiogenesis, via the lowering of VEGF levels as well as other cytokines that drive tumor growth.

The linkage between PI3K/mTOR pathway signaling and androgen receptor activity has been reported that points out the therapeutic synergy between combination PI3K/mTOR pathway inhibition and androgen receptor signaling inhibition. PI3K/AKT/mTOR and AR pathways have been shown to cross-regulate through several reciprocal inhibitory loops (see e.g., Shoring et al. (2020) Int. J. Mol. Sci. 21:4507). The interconnection between the PI3K pathway and AR signaling is also discussed in, for example, Crumbaker et al. (2017) Cancers (Basel) 9:34; Raith et al. (2023) Int. J. Mol. Sci. 24:2289; and Tortorella et al. (2023) Int. J. Mol. Sci. 24:2046. Consequently, the PI3K-AKT-mTOR pathway can be inadvertently activated in response to androgen/AR-directed therapies, and vice versa PI3K-AKT-mTOR pathway inhibition can augment AR signaling, leading to therapeutic resistance.

Furthermore, Shorning et al. (2020), supra, report that human patient samples (both primary tumor and bone metastases), human prostate cancer cell lines and transgenic mouse models of prostate cancer have consistently demonstrated that PI3K/AKT/mTOR signaling is increased in response to androgen/AR-directed blockade. Mechanistically, it is reported that inhibiting AR signaling reduces expression of the AR target gene FK506-binding protein-5 (FKBP5), which leads to PHLPP destabilization and reduced PHLPP-mediated dephosphorylation of AKT at Ser473 to promote AKT signaling. Thus, compensatory activation of the PI3K-AKT-mTOR pathway in response to androgen/AR pathway inhibition can facilitate castration-resistant prostate cancer (CRPC) growth.

Additionally, PI3K-AKT-mTOR pathway inhibition is associated with augmented AR signaling that can contribute to drug resistance and promote prostate cancer progression. PI3K/mTOR inhibition has been shown to activate AR signaling in human xenograft and transgenic mouse models of prostate cancer, and that co-treatment with a weak PI3K/mTOR inhibitor, BEZ235, and the antiandrogen MDV3100 (enzalutamide) significantly reduced tumor burden relative to monotherapy (Carver et al. (2011) Cancer Cell 19:575-586).

Taken together, these findings support the rationale for combining pharmacological inhibition of the AR and PI3K-AKT-mTOR cascades with a drug such as gedatolisib to treat prostate cancer in the clinic.

The safety, tolerability, pharmacokinetics and preliminary activity of gedatolisib in solid tumors was tested in a Phase 1 trial in combination with the chemotherapeutic agents carboplatin and paclitaxel (Colombo et al. (2021) Clin. Cancer Res. 27:5012-5019). The results demonstrated that gedatolisib was well tolerated and showed preliminary efficacy in combination with the chemotherapeutic agents.

More recent clinical studies using gedatolisib have focused on treatment of breast cancer. For example, a Phase 1b trial in patients with metastatic triple-negative breast cancer using gedatolisib in combination with an antibody-drug conjugate that targets the cell-surface PTK7 protein, cofetuzumab pelidotin, reported that the combination was well tolerated and showed promising activity (Radovich et al. (2022) Clin. Cancer Res. 28:3235-3241). Another Phase I study examining gedatolisib in combination with the chemotherapeutic agents docetaxel, cisplatin and/or dacomitinib in triple negative breast cancer also reported tolerability and promising activity (Curigliano et al. (2023) Br. J. Cancer 128:30-41). Gedatolisib treatment in hormone receptor-positive, HER2-negative advanced breast cancer also is being studied in combination with endocrine therapy and the CDK 4/6 inhibitor palbociclib, initial results of which are reported in Layman et al. (2024) Lancet Oncol. 25:474-487).

While certain advances have been made in the treatment of cancer using gedatolisib, additional treatment regimens for use of gedatolisib, and additional indications for treatment of cancer, are still needed in the art.

### BRIEF SUMMARY

This disclosure provides methods of treating hormonally driven disorders other than breast cancer in a subject (e.g., a human subject) by administering gedatolisib to the subject in combination with hormone therapy. In certain embodiments, the method further comprises treating the subject (e.g., a human subject) with one or more additional therapies effective in the hormonally driven disorder.

Accordingly, in one aspect, the disclosure pertains to a method of treating a hormonally driven disorder in a human subject, the method comprising administering to a human subject with a hormonally driven disorder:
gedatolisib, or a pharmaceutically acceptable salt, solvate, or ester thereof, at a dosage of 120-300 mg weekly for at least two weeks; and
a hormone therapy for at least two weeks;
wherein the hormonally driven disorder is not breast cancer.

In embodiments, the hormonally driven disorder is prostate cancer. In embodiments, the prostate cancer is diagnosed as metastatic hormone sensitive prostate cancer, non-metastatic castration resistant prostate cancer, or metastatic castration resistant prostate cancer. In other embodiments, the prostate cancer has become hormone therapy resistant and has progressed during treatment with an ARsi at time of administering gedatolisib, or a pharmaceutically acceptable salt, solvate, or ester thereof. In other embodiments, the prostate cancer remains hormone sensitive at the time of administering gedatolisib, or a pharmaceutically acceptable salt, solvate, or ester thereof.

In embodiments, such as in prostate cancer, the hormone therapy comprises administering to the subject an androgen receptor signaling inhibitor (ARsi). In embodiments, the ARsi is a non-steroidal androgen receptor inhibitor (ARi). In embodiments, the non-steroidal ARi is selected from the group consisting of darolutamide, apalutamide, bicalutamide, enzalutamide, flutamide, nilutamide and topilutamide. In embodiments, the non-steroidal ARi is darolutamide. In embodiments, darolutamide is orally administered at a dosage of 600 mg twice daily.

In other embodiments, the hormone therapy comprises administering to the subject a steroidal anti-androgen, an androgen synthesis inhibitor (for example any antagonist of the biochemical synthetic pathway from cholesterol to testosterone or dihydrotestosterone) or an androgen receptor pathway inhibitor. Non-limiting examples of suitable steroidal anti-androgens, androgen synthesis inhibitors or androgen receptor pathway inhibits are disclosed herein.

In other embodiments, the hormonally driven disorder is endometrial cancer, uterine cancer or ovarian cancer.

In other embodiments, the hormonally driven disorder is an endometrial disorder. Non-limiting examples of endometrial disorders include endometriosis, endometrial hyperplasia, endometrial intraepithelial neoplasia (EIN), intrauterine adhesions (IUA), adenomyosis, endometritis, endometrial and/or uterine polyps and Asherman syndrome. In an embodiment, the endometrial disorder is endometriosis.

In embodiments, gedatolisib, or a pharmaceutically acceptable salt, solvate, or ester thereof, is administered at a dosage of 100 mg weekly, or at a dosage of 120 mg weekly, or at a dosage of 150 mg weekly, or at a dosage of 180 mg weekly, or at a dosage of 210 mg weekly, or at a dosage of 240 mg weekly. In embodiments, gedatolisib, or a pharmaceutically acceptable salt, solvate, or ester thereof, is administered intravenously. Suitable pharmaceutical formulations for administration of gedatolisib, or a pharmaceutically acceptable salt, solvate, or ester thereof (e.g., i.v. administration) are described herein.

In embodiments, gedatolisib, or a pharmaceutically acceptable salt, solvate, or ester thereof, and the hormone therapy are administered for more than two weeks, e.g., for at least three weeks, for at least four weeks, for at least five weeks, for at least six weeks, for at least seven weeks, for at least eight weeks or longer, as described further herein. In embodiments, gedatolisib, or a pharmaceutically acceptable salt, solvate, or ester thereof, is administered weekly. In other embodiments, gedatolisib, or a pharmaceutically acceptable salt, solvate, or ester thereof, is administered according to a three week on/one week off treatment schedule, as described further herein.

In embodiments, the method further comprises treating the human subject with one or more additional therapies effective in the hormonally driven disorder. In an embodiment, the one or more additional therapies comprise administering to the subject one or more chemotherapeutic drugs. In an embodiment, the one or more additional therapies comprise administering to the subject one or more immune checkpoint inhibitors. In an embodiment, the one or more additional therapies comprise administering to the subject one or more epigenetic inhibitors. In an embodiment, the one or more additional therapies comprise one or more surgical treatments.

In another aspect, the disclosure pertains to a method of treating prostate cancer in a human subject, the method comprising administering to a human subject with prostate cancer:
gedatolisib, or a pharmaceutically acceptable salt, solvate, or ester thereof, at a dosage of 100-300 mg weekly for at least two weeks; and
a hormone therapy for at least two weeks.

For example, disclosure provides a method of treating prostate cancer in a human subject, the method comprising administering to a human subject with prostate cancer:
gedatolisib, or a pharmaceutically acceptable salt, solvate, or ester thereof, at a dosage of 120-300 mg weekly; and
darolutamide at a dosage of 600 mg twice daily.

In embodiments, the prostate cancer has become hormone resistant and has progressed during treatment with an ARsi at time of administering gedatolisib, or a pharmaceutically acceptable salt, solvate, or ester thereof. In other embodiments, the prostate cancer remains hormone driven at the time of administering gedatolisib, or a pharmaceutically acceptable salt, solvate, or ester thereof.

In embodiments, the hormone therapy comprises administering to the subject a non-steroidal androgen receptor inhibitor (ARi). In embodiments, the non-steroidal ARi is selected from the group consisting of darolutamide, apalutamide, bicalutamide, enzalutamide, flutamide, nilutamide and topilutamide. In embodiments, the non-steroidal ARi is darolutamide. In embodiments, darolutamide is orally administered at a dosage of 600 mg twice daily.

In other embodiments, the hormone therapy comprises administering to the subject a steroidal anti-androgen, an androgen synthesis inhibitor or an androgen receptor pathway inhibitor.

In embodiments, gedatolisib, or a pharmaceutically acceptable salt, solvate, or ester thereof, is administered at a dosage of 100 mg weekly, or at a dosage of 120 mg weekly, or at a dosage of 150 mg weekly, or at a dosage of 180 mg weekly, or at a dosage of 210 mg weekly, or at a dosage of 240 mg weekly. In embodiments, gedatolisib, or a pharmaceutically acceptable salt, solvate, or ester thereof, is administered intravenously.

In embodiments, gedatolisib, or a pharmaceutically acceptable salt, solvate, or ester thereof, and the hormone therapy are administered for more than two weeks, e.g., for at least three weeks, for at least four weeks, for at least five weeks, for at least six weeks, for at least seven weeks, for at least eight weeks or longer, as described further herein. In embodiments, gedatolisib, or a pharmaceutically acceptable salt, solvate, or ester thereof, is administered weekly. In other embodiments, gedatolisib, or a pharmaceutically acceptable salt, solvate, or ester thereof, is administered according to a three week on/one week off treatment schedule, as described further herein.

In embodiments, the method of treating prostate cancer further comprises treating the human subject with one or more additional therapies effective in prostate cancer, such as the additional therapies described above. In embodiments, the one or more additional therapies comprise one or more surgical treatments that lower testosterone.

In another embodiment, the disclosure pertains to a method of treating a hormonally driven disorder other than breast cancer in a human subject comprising:
administering to a human subject with a hormonally driven disorder other than breast cancer:
gedatolisib, or a pharmaceutically acceptable salt, solvate, or ester thereof, at a dosage of 100-300 mg once a week for an administration period of three weeks;
discontinuing administration of gedatolisib, or pharmaceutically acceptable salt, solvate, or ester thereof, for a discontinuation period of one week; and
resuming administration of gedatolisib, or pharmaceutically acceptable salt, solvate, or ester thereof, once a week following the discontinuation period,
wherein the administration period of three weeks and the discontinuation period of one week constitutes a cycle, wherein the cycle is repeated for at least two cycles; and
a hormone therapy that is administered for the at least two cycles.

In another embodiment, the disclosure pertains to a method of treating prostate cancer in a human subject comprising:
administering to a human subject with prostate cancer:
(a) gedatolisib, or a pharmaceutically acceptable salt, solvate, or ester thereof, at a dosage of 100-300 mg once a week for an administration period of three weeks;
discontinuing administration of gedatolisib, or pharmaceutically acceptable salt, solvate, or ester thereof, for a discontinuation period of one week; and
resuming administration of gedatolisib, or pharmaceutically acceptable salt, solvate, or ester thereof, once a week following the discontinuation period; and
(b) darolutamide at a dosage of 600 mg twice daily for an administration period of three weeks;
discontinuing administration of darolutamide for a discontinuation period of one week; and
resuming administration of darolutamide at a dosage of 600 mg twice daily following the discontinuation period;
wherein the administration period of three weeks and the discontinuation period of one week constitutes a cycle, wherein the cycle is repeated for at least two cycles; and
wherein the discontinuation period for administering gedatolisib, or a pharmaceutically acceptable salt, solvate, or ester thereof, and the discontinuation period for administering darolutamide are concurrent.

In another embodiment, the disclosure pertains to a method of treating an endometrial disorder in a human subject comprising:
administering to a human subject with an endometrial disorder:
(a) gedatolisib, or a pharmaceutically acceptable salt, solvate, or ester thereof, at a dosage of 100-300 mg once a week for an administration period of three weeks;
discontinuing administration of gedatolisib, or pharmaceutically acceptable salt, solvate, or ester thereof, for a discontinuation period of one week; and
resuming administration of gedatolisib, or pharmaceutically acceptable salt, solvate, or ester thereof, once a week following the discontinuation period;
(b) a hormone therapy; and
(c) a CDK4/6 inhibitor for an administration period of three weeks;
discontinuing administration of the CDK4/6 inhibitor for a discontinuation period of one week; and
resuming administration of the CDK4/6 inhibitor following the discontinuation period;
wherein the administration period of three weeks and the discontinuation period of one week constitutes a cycle, wherein the cycle is repeated for at least two cycles;
wherein the discontinuation period for administering gedatolisib, or a pharmaceutically acceptable salt, solvate, or ester thereof, and the discontinuation period for administering the CDK 4/6 inhibitor are concurrent; and
wherein the endometrial disorder is selected from the group consisting of endometriosis, endometrial hyperplasia, endometrial carcinoma, endometrial intraepithelial neoplasia (EIN), intrauterine adhesions (IUA), adenomyosis, endometritis, endometrial and/or uterine polyps and Asherman syndrome.

In yet another aspect, the disclosure pertains to a method of treating an endometrial disorder in a human subject, the method comprising administering to a human subject with an endometrial disorder:
gedatolisib, or a pharmaceutically acceptable salt, solvate, or ester thereof, at a dosage of 100-300 mg weekly for at least two weeks; and
a hormone therapy for at least two weeks;
wherein the endometrial disorder is selected from the group consisting of endometriosis, endometrial hyperplasia, endometrial intraepithelial neoplasia (EIN), intrauterine adhesions (IUA), adenomyosis, endometritis, endometrial and/or uterine polyps and Asherman syndrome.

In embodiments, the endometrial disorder is endometriosis.

In embodiments, the hormone therapy comprises administering to the subject a selective estrogen receptor modulator (SERM), non-limiting examples of which include tamoxifen, raloxifene, toremifene, ospemifene, bazedoxifene, ormeloxifene, lasofoxifene, cyclofenil, clomifene, broparestrol and anordin. In an embodiment, the SERM is tamoxifen.

In embodiments, the hormone therapy comprises administering to the subject a selective estrogen receptor degrader (SERD), non-limiting examples of which include fulvestrant, elacestrant, amcenestrant, camizestrant, giredestrant, imlunestrant, vepdegestrant, palazestrant, LSZ102, rintodestrant, SHR9549, H3B-5942, ZN-c5, and brilanestrant. In an embodiment, the SERD is fulvestrant.

In embodiments, the hormone therapy comprises administering to the subject an aromatase inhibitor, non-limiting examples of which include letrozole, anastrozole and exemestane. In embodiments, the aromatase inhibitor is letrozole.

In embodiments, gedatolisib, or a pharmaceutically acceptable salt, solvate, or ester thereof, is administered at a dosage of 100 mg weekly, or at a dosage of 120 mg weekly, or at a dosage of 150 mg weekly, or at a dosage of 180 mg weekly, or at a dosage of 210 mg weekly, or at a dosage of 240 mg weekly. In embodiments, gedatolisib, or a pharmaceutically acceptable salt, solvate, or ester thereof, is administered intravenously.

In embodiments, gedatolisib, or a pharmaceutically acceptable salt, solvate, or ester thereof, and the hormone therapy are administered for more than two weeks, e.g., for at least three weeks, for at least four weeks, for at least five weeks, for at least six weeks, for at least seven weeks, for at least eight weeks or longer, as described further herein. In embodiments, gedatolisib, or a pharmaceutically acceptable salt, solvate, or ester thereof, is administered weekly. In other embodiments, gedatolisib, or a pharmaceutically acceptable salt, solvate, or ester thereof, is administered according to a three week on/one week off treatment schedule, as described further herein.

In embodiments, the method of treating an endometrial disorder (e.g., endometriosis) further comprises treating the human subject with one or more additional therapies effective in the endometrial disorder, such as the additional therapies described above.

Other features and advantages disclosed herein will be apparent from the following detailed description and claims.

### BRIEF DESCRIPTION OF THE DRAWINGS

The accompanying FIGURES, which are incorporated herein and form a part of the specification, illustrate some, but not the only or exclusive, example embodiments and/or features. It is intended that the embodiments and FIGURES disclosed herein are to be considered illustrative rather than limiting.

FIG. 1A shows preliminary results of the randomized BOIN Design where subjects received 120 mg or 180 mg gedatolisib. The “historical data1” is described in Morris NEJM 2024; Abbreviations: rPFS6-six-month radiographic progression free survival probability. FIG. 1B shows a summary of the radiological progression free survival (rPFS6) and a summary of adverse events for treated subjects. FIG. 1C shows a Kaplan Meier curve for rPFS for the phase I study denoting each of arm 1 and arm 2. FIG. 1D shows a Kaplan Meier curve for combined rPFS for both arms of the phase I study.

### DETAILED DESCRIPTION

Described herein are treatment regimens for use of gedatolisib in combination with hormone therapy, and optionally one or more additional therapies, in hormonally driven disorders other than breast cancer.

In order that the present description may be more readily understood, certain terms are first defined. Additional definitions are set forth throughout the detailed description. Unless defined otherwise, all technical and scientific terms used herein have the same meaning as commonly understood by one of ordinary skill in the art.

As used herein, the singular forms “a”, “an” and “the” include plural referents unless the context clearly dictates otherwise. The use of “or” or “and” means “and/or” unless stated otherwise.

The term “about” as used herein when referring to a measurable value such as an amount, a temporal duration and the like, is encompasses variations of up to +10% from the specified value. Unless otherwise indicated, all numbers expressing quantities of ingredients, properties such as molecular weight, reaction conditions, etc., used herein are to be understood as being modified by the term “about”.

The term “inhibition” or “reduction” as used herein, refers to any statistically significant decrease in biological activity, including partial and full blocking of the activity. For example, “inhibition” or “reduction” can refer to a statistically significant decrease of about 10%, 20%, 30%, 40%, 50%, 60%, 70%, 80%, 90%, 95%, 96%, 97%, 98%, 99%, or 100% in biological activity. The terms “inhibits” or “blocks” (e.g., referring to inhibition/blocking of binding or activity) are used interchangeably and encompass both partial and complete inhibition/blocking.

As used herein, the term “subject” includes any human or non-human animal. For example, the methods and compositions described herein can be used to treat a subject (e.g., a human patient) having a hormonally driven disorder.

A “therapeutically effective amount” means an amount of gedatolisib, or other active agent, set forth herein that, when administered to a subject, is effective in producing a therapeutic effect.

As used herein, “administering” refers to the physical introduction of a composition comprising a therapeutic agent to a subject, using any of the various methods and delivery systems known to those skilled in the art. Preferred routes of administration for the therapeutic agents described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intra-arterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually, or topically. Administering can also be performed, for example, once, a plurality of times, and/or over one or more extended periods.

As used herein, the terms “treatment,” “treating”, “treat”, or the like, mean to alleviate or reduce the severity of at least one symptom or indication, to eliminate the causation of symptoms either on a temporary or permanent basis, or to obtain beneficial or desired clinical results. Beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. Treatment may result in a partial response (PR) or a complete response (CR).

The term “hormone therapy” or “hormonal therapy” denotes a treatment which targets hormone signaling, e.g. hormone signaling inhibition, hormone receptor inhibition, use of hormone receptor agonists or antagonists, use of scavenger- or orphan receptors, use of hormone derivatives and interference with hormone production. Non-limiting examples of hormones that can be a target of hormone therapy include estrogen, testosterone, progesterone, steroid, glucocorticoids and thyroid hormone.

The term “failed prior treatment” denotes that a subject who has been undergoing treatment for cancer has experienced a progression of the cancer during the treatment, e.g., within a specified time period of treatment (such as within twelve months, or six months of the onset of treatment). The term “progression” of a cancer denotes increased growth and/or spread (e.g., metastasis), typically measured by means established in the art for assessing cancer growth and/or spread, including but not limited to bodily scans (e.g., MRI scans, PET scans, CAT scans and the like), biopsies and/or measurement of biomarkers. In some embodiments, progression is defined as at least 20% increase in the sum of the diameters of the target measurable lesions (e.g., tumors) above the smallest sum observed, or over the baseline sum of diameters, with a minimum absolute increase of at least 5 mm.

The term “therapy modality”, “therapy mode”, “schedule”, “regimen” as well as “therapy regimen” refers to a timely sequential or simultaneous administration of anti-tumor, and/or anti vascular, and/or immune stimulating, and/or blood cell proliferative agents, and/or radiation therapy, and/or hyperthermia, and/or hypothermia for cancer therapy. The administration of these can be performed in an adjuvant and/or neoadjuvant mode. The composition of such “protocol” may vary in the dose of the single agent, timeframe of application and frequency of administration within a defined therapy window.

The term “cytotoxic chemotherapy” refers to various treatment modalities affecting cell proliferation and/or survival. The treatment may include administration of alkylating agents, antimetabolites, anthracyclines, plant alkaloids, topoisomerase inhibitors, and other antitumor agents, including monoclonal antibodies and kinase inhibitors. In particular, the cytotoxic treatment may relate to a taxane treatment. Taxanes are plant alkaloids which block cell division by preventing microtubule function. The prototype taxane is the natural product paclitaxel, originally known as Taxol and first derived from the bark of the Pacific Yew tree. Docetazel is a semi-synthetic analogue of paclitaxel. Taxanes enhance stability of microtubules, preventing the separation of chromosomes during anaphase.

Various aspects of the disclosure are described in further detail in the following subsections.

### I. Hormonally Driven Disorders

In the methods of the disclosure, gedatolisib is used in combination with hormone therapy in the treatment of a hormonally driven disorder other than breast cancer.

In embodiments, the hormonally driven disorder is a hormonally driven cancer other than breast cancer. Non-limiting examples of hormonally driven cancers other than breast cancers include prostate cancers, endometrial cancers, uterine cancers and ovarian cancers.

In embodiments, the hormonally driven cancer is metastatic cancer as determined by imaging. Exemplary imaging modalities comprise computed tomography (CT), magnetic resonance imaging (MRI), or technetium 99m-methyl diphosphonate (99mTc-MDP) bone scintigraphy.

In embodiments, the hormonally driven cancer being treated according to a method of the disclosure remains hormonally driven at the time of gedatolisib administration. In other embodiments, the hormonally driven cancer being treated according to a method of the disclosure has become hormonally resistant at the time of gedatolisib administration (e.g., due to failure of a prior hormone therapy).

In other embodiments, the hormonally driven disorder is an endometrial disorder. Non-limiting examples of endometrial disorders include endometriosis, endometrial hyperplasia, endometrial intraepithelial neoplasia (EIN), intrauterine adhesions (IUA), adenomyosis, endometritis, endometrial and/or uterine polyps and Asherman syndrome. In an embodiment, the endometrial disorder is endometriosis.

In embodiments, the hormonally driven disorder is prostate cancer. Prostate cancer is the most prevalent cancer in men, and one of the most prevalent cancers overall in the United States (US). While tumors confined to the prostate or regional lymph nodes have a 5-year survival rate of 100%, only 32% of men with metastatic disease survive for five years. Androgen deprivation therapy (ADT) with or without concomitant radiation is the current first-line treatment for patients with castration-naïve metastatic disease. ADT blocks the production of androgens in the testes using luteinizing hormone-releasing hormone (LHRH) agonists such as leuprolide or antagonists such as degarelix (de Bono et al. (2011) N Engl J Med. 364:1995-2005; Scher et al. (2012) N Engl J Med. 367:1187-1197; Shorning et al. (2020) Int J Mol Sci. 21:4507).

Though most patients respond favorably to ADT initially, their disease often metastasizes to other organs while remaining hormone-sensitive (mHSPC) or becomes castration-resistant and emerges as non-metastatic or metastatic castration-resistant prostate cancer (nmCRPC) (Crawford et al. (2018) Prostate Cancer Prostatic Dis. 22:24-38). Despite the recent approvals of several targeted therapies to treat mHSPC, nmCRPC or mCRPC, such as next-generation androgen receptor (AR) inhibitors like enzalutamide, abiraterone, apalutamide, and darolutamide, and poly ADP ribose polymerase (PARP) inhibitors like olaparib or rucaparib for patients with BRCA mutations, mHSPC, nmCRPC, or mCRPC typically remains uncurable (Crawford et al. (2018) Prostate Cancer Prostatic Dis. 22:24-38; Shorning et al. (2020) Int J Mol Sci. 21:4507).

For patients with newly diagnosed mHSPC, nmCRPC or mCRPC lacking a BRCA mutation, next-generation AR signaling inhibitors, abiraterone, enzalutamide, apalutamide, and darolutamide, are among the current standard of care first-line treatments. For patients with newly diagnosed mCRPC, the chemotherapy, docetaxel, is an additional standard of care first-line treatment option. For mCRPC patients who received docetaxel as first-line therapy, abiraterone and enzalutamide are the preferred second-line treatment options. For mHSPC, nmCRPC, or mCRPC patients who received an AR signaling inhibitor (ARsi) as first-line therapy, docetaxel is the preferred option, but treatment with a different ARsi than the patient received initially is also a recommended option. However, the median progression-free survival reported in randomized clinical studies for these second-line regimens is only 5-7 months, highlighting the need for more effective treatment options. In embodiments, the prostate cancer is selected from the group consisting of adenocarcinoma of the prostate, transitional cell carcinoma of the prostate, squamous cell carcinoma of the prostate and small cell prostate cancer. In embodiments, the prostate cancer is adenocarcinoma of the prostate. In embodiments the prostate cancer is transitional cell carcinoma of the prostate. In embodiments the prostate cancer is squamous cell carcinoma of the prostate. In embodiments the prostate cancer is small cell prostate cancer.

In embodiments, a subject treated with a method of the disclosure has prostate-specific antigen (PSA) progression. PSA progression refers to a minimum of 2 rising PSA levels with a minimum of a 1-week interval between each determination.

In embodiments, a subject treated with a method of the disclosure has a minimum PSA of at least about 1.0 ng/ml, 1.5 ng/mL, 2.0 ng/mL, or 2.5 ng/mL. In embodiments, a subject treated with a method of the disclosure has a minimum PSA of at least about 1.0 ng/ml.

Example 1 describes in detail a Phase 1/2 trial of gedatolisib in combination with hormone therapy in the treatment of mCRPC.

### II. Gedatolisib

Gedatolisib, also known as 1-(4-{[4-(Dimethylamino)piperidin-1-yl]carbonyl}phenyl)-3-[4-(4,6-dimorpholin-4-yl-1,3,5-triazin-2-yl)phenyl]urea, is a multi-target inhibitor that addresses all four class I PI3K isoforms and the mTOR complexes mTORC1 and mTORC2 to induce comprehensive blockade of the PI3K/AKT/mTOR (“PAM”) pathway.

The chemical synthesis of gedatolisib is disclosed in U.S. Pat. Nos. 8,039,469; 8,217,036; 8,445,486; 8,575,159; 8,748,421; 8,859,542; 9,174,963; 10,022,381, which are hereby incorporated by reference in their entirety. Gedatolisib may be prepared in crystalline form and is chemically and physically stable at 25° C. and 60% Relative Humidity (RH) for up to 3 years in this form. As a free base, gedatolisib is insufficiently water soluble. To allow the preparation of an aqueous solution formulation suitable for intravenous, topical, injectable intraocular, or other parenteral administration at the therapeutic dosage levels small amounts of acid are required. Accordingly, formulations that allow for therapeutic dosage levels have been developed, including formulations comprising lactic acid and cyclodextrin, as described further below.

In various embodiments, gedatolisib, or a pharmaceutically acceptable salt, solvate, or ester thereof, is administered at a dosage of 100-300 mg per dose, administered weekly. In embodiments, gedatolisib, or a pharmaceutically acceptable salt, solvate, or ester thereof is administered at a dosage of at least about or at most about: 100 mg, 102 mg, 104 mg, 106 mg, 108 mg, 110 mg, 112 mg, 114 mg, 116 mg, 118 mg, 120 mg, 122 mg, 124 mg, 126 mg, 128 mg, 130 mg, 132 mg, 134 mg, 136 mg, 138 mg, 140 mg, 142 mg, 144 mg, 146 mg, 148 mg, 150 mg, 152 mg, 154 mg, 156 mg, 158 mg, 160 mg, 162 mg, 164 mg, 166 mg, 168 mg, 170 mg, 172 mg, 174 mg, 176 mg, 178 mg, 180 mg, 182 mg, 184 mg, 186 mg, 188 mg, 190 mg, 192 mg, 194 mg, 196 mg, 198 mg, 200 mg, 202 mg, 204 mg, 206 mg, 208 mg, 210 mg, 212 mg, 214 mg, 216 mg, 218 mg, 220 mg, 222 mg, 224 mg, 226 mg, 228 mg, 230 mg, 232 mg, 234 mg, 236 mg, 238 mg, or 240 mg. In embodiments, gedatolisib, or a pharmaceutically acceptable salt, solvate, or ester thereof is administered at a dosage of about 100 mg-150 mg, 125 mg-175 mg, 150 mg-200 mg, 175 mg-225 mg, or 200 mg-240 mg. In embodiments, any of the aforementioned dosages or dosage ranges can be administered hourly, daily, weekly, monthly, or annually.

In embodiments, gedatolisib, or a pharmaceutically acceptable salt, solvate, or ester thereof, is administered at a dosage of 100 mg weekly, or at a dosage of 120 mg weekly, or at a dosage of 150 mg weekly, or at a dosage of 180 mg weekly, or at a dosage of 210 mg weekly or at a dosage of 240 mg weekly.

In embodiments, gedatolisib is administered intravenously (i.v.). In embodiments, gedatolisib is prepared as an aqueous pharmaceutical formulation suitable for i.v. administration. In embodiments, gedatolisib is prepared as a lyophilized formulation that is reconstituted as an aqueous formulation prior to i.v. administration.

In embodiments, gedatolisib is administered on a weekly basis for at least two weeks. In other embodiments, gedatolisib is administered on a weekly basis for at least three weeks, at least four weeks, at least five weeks, at least six weeks, at least seven weeks, at least eight weeks, at least one month, at least two months, at least three months, at least five months, at least six months or longer.

In embodiments, gedatolisib is administered according to a treatment schedule of three weeks on/one week off, which constitutes one cycle, wherein treatment lasts for at least two cycles. Thus, gedatolisib can be administered according to a treatment schedule of weekly gedatolisib for three weeks, followed by one week of no treatment, followed by weekly gedatolisib for three weeks, followed by one week of no treatment.

1-(4-{[4-(Dimethylamino)piperidin-1-yl]carbonyl}phenyl)-3-[4-(4,6-dimorpholin-4-yl-1,3,5-triazin-2-yl)phenyl]urea, has the chemical structure:

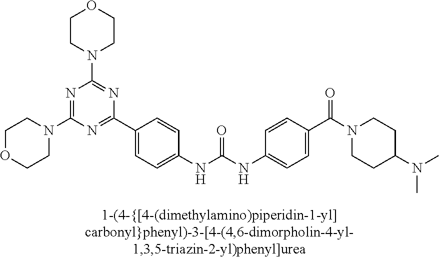 [SMILES: CN(C)C1CCN(C(=O)c2ccc(NC(=O)Nc3ccc(-c4nc(N5CCOCC5)nc(N5CCOCC5)n4)cc3)cc2)CC1]

Gedatolisib is a small molecule, which inhibits Phosphatidylinositol-3 kinase (PI3K) and Mammalian Target of Rapamycin (mTOR). Phosphatidylinositol-3 kinase is an enzyme that phosphorylates the 3-position of the inositol ring of phosphatidylinositol (D. Whitman et al., (1988)) to make a critical signaling phospholipid, PIP3, known to be extensively involved in activating a plethora of protein activities. Pluralities of PI3K subtypes exist, with three major subtypes of PI3Ks having now been identified based on their in vitro substrate specificity. These three are designated class I (a & b), class II, and class III. Gedatolisib has low nanomolar potency for the p110a, p110b, p110g and p110d isoforms of PI3K, as well as for mTORC1 and mTORC2.

Mammalian Target of Rapamycin (mTOR) is a cell-signaling protein that regulates the response of tumor cells to nutrient supply and growth factors to control protein translation, cancer cell proliferation, and tumor blood supply through effects on Vascular Endothelial Growth Factor (VEGF). Some mTOR inhibitors like gedatolisib bind directly to the mTOR kinase and compete with ATP binding thus blocking mTOR kinase activity. This has at least two important effects. First, mTOR is a downstream mediator of the PI3K/Akt pathway, having both forward signaling properties and feedback properties on the pathway. The PI3K/Akt pathway has been demonstrated to be over activated in numerous cancers and may account for the widespread response from various cancers to mTOR inhibitors. The PI3K/Akt pathway is demonstrated to be over activated in numerous metabolic diseases as well, consuming increased levels of oxygen and glucose chronically and furthermore creating a state of metabolic dysfunction within the disease tissue such as exemplified by increased cytokine production, hypoxia, and low pH. The over-activation of signaling nodes upstream in the PI3K/Akt pathway can cause mTOR kinase to be over activated as well. However, in the presence of mTOR inhibitors, mTOR signaling processes are blocked. The blocking effect prevents mTOR from signaling to downstream pathways that control critical cell functions such as reducing protein translation, cell cycling, glucose consumption, and ultimately affect diseased cell growth and proliferation. One of the major effects of PI3K/Akt pathway inhibition by reducing protein translation is reducing cytokine production and specifically anti-angiogenesis, via the lowering of the production of VEGF and other angiogenic protein production levels (e.g., Ang2, FGF, PDGF).

As used herein the terms “gedatolisib” and “1-(4-{[4-(Dimethylamino)piperidin-1-yl]carbonyl}phenyl)-3-[4-(4,6-dimorpholin-4-yl-1,3,5-triazin-2-yl)phenyl]urea” refer to the same compound and may be used interchangeably and are intended to encompass the pharmaceutically acceptable salts, solvates or esters of gedatolisib.

Representative “pharmaceutically acceptable salts” include but are not limited to, e.g., water-soluble and water-insoluble salts or their acid forms, such as 2,3, or 4 carbon mono and di-carboxylates or mono and di-carboxylic acids, acetate, aluminum, amsonate (4,4-diaminostilbene-2,2-disulfonate), benzathine (N,N′-dibenzylethylenediamine), benzenesulfonate, benzoate, bicarbonate, bismuth, bisulfate, bitartrate, borate, bromide, butyrate, calcium, calcium edetate, camsylate (camphorsulfonate), carbonate, chloride, choline, citrate, clavulariate, diethanolamine, dihydrochloride, diphosphate, edetate, edisylate (camphorsulfonate), esylate (ethanesulfonate), ethylenediamine, fumarate, gluceptate (glucoheptonate), gluconate, glucuronate, glutamate, hexafluorophosphate, hexylresorcinate, hydrabamine (N,N′-bis (dehydroabietyl)ethylenediamine), hydrobromide, hydrochloride, hydroxynaphthoatc, 1-hydroxy-2-naphthoate, 3-hydroxy-2-naphthoate, iodide, isothionate (2-hydroxyethanesulfonate), lactate, lactobionate, laurate, lauryl sulfate, lithium, magnesium, malate, maleate, mandelate, meglumine (1-deoxy-1-(methylamino)-D-glucitol), mesylate, methyl bromide, methylnitrate, methylsulfate, mucate, napsylate, nitrate, N-methylglucamine ammonium salt, oleate, oxalate, palmitate, pamoate (4,4′-methylenebis-3-hydroxy-2-naphthoate, or embonate), pantothenate, phosphate, picrate, polygalacturonate, potassium, propionate, p-toluenesulfonate, salicylate, sodium, stearate, subacetate, succinate, sulfate, sulfosaliculate, suramate, tannate, tartrate, teoclate (8-chloro-3,7-dihydro-1,3-dimethyl-1H-purine-2,6-dione), trieth iodide, tromethamine (2-amino-2-(hydroxymethyl)-1,3-propanediol), valerate, and zinc salts.

Pharmaceutically acceptable esters include, but are not limited to, alkyl, alkenyl, alkynyl, aryl, heteroaryl, cycloalkyl and heterocyclyl esters of acidic groups, including, but not limited to, carboxylic acids, phosphoric acids, phosphinic acids, sulfonic acids, sulfinic acids, and boronic acids.

Pharmaceutical acceptable solvates and hydrates are complexes of a compound with one or more solvent or water molecules, or 1 to about 100, or 1 to about 10, or one to about 2, 3 or 4, solvent or water molecules.

Pharmaceutical formulations comprising therapeutic dosage levels of gedatolisib are known in the art and include aqueous intravenous formulations, as well as nanoparticle formulations.

PCT application publication WO2016/097949 discloses aqueous intravenous formulations of gedatolisib with lactic acid and/or orthophosphoric acid, which form clear, particulate free solutions. The formulations include gedatolisib, lactic acid, and water. The gedatolisib has a concentration in the solution less than 6 mg/ml (preferably about 5 mg/ml), and there is sufficient lactic acid present to provide a clear solution (preferably at least 2.5 mole equivalents). The gedatolisib forms a 1:1 (mole equivalent) lactate salt with lactic acid. Therefore, the formulations can be prepared using the gedatolisib free base or using a lactic acid salt of gedatolisib.

The formulations with orthophosphoric acid include gedatolisib, orthophosphoric acid, and water. The gedatolisib is present at a solution concentration of less than 4 mg/ml (preferably from 3.0 to 3.5 mg/ml) and sufficient orthophosphoric acid is present to provide a clear solution (preferably at least 5 mole equivalents).

Formulations including gedatolisib and cyclodextrins are disclosed in PCT application publication WO 2019/234632. The pharmaceutical aqueous formulations include gedatolisib, or a pharmaceutically acceptable organic or inorganic acid salt thereof, a pharmaceutically acceptable organic or inorganic acid, which is not a sulphonic acid, a pharmaceutically acceptable beta- or gamma-cyclodextrin and water. In an embodiment, the beta-cyclodextrin is (2-Hydroxypropyl)-β-cyclodextrin (CAS number 128446-35-5, EC Number 420-920-1, also sometimes known as Cavitron). The gedatolisib is present at a solution concentration of at least 6 mg/ml and the solutions are clear.

The pharmaceutically acceptable organic acid used (including for a salt thereof) are 2,3, or 4 carbon mono and di-carboxylates or mono and di-carboxylic acids, lactic acid, tartaric acid, malic acid, citric acid, succinic acid, acetic acid or maleic acid. The acid may be used in its racemic form, or as a single stereoisomeric form (or mixtures thereof), where applicable. Examples of a pharmaceutically acceptable beta-cyclodextrin are 2-hydroxypropyl-beta-cyclodextrin and sulphobutylether-β-cyclodextrin (SBECD). Examples of such a pharmaceutically acceptable gamma-cyclodextrin are gamma-cyclodextrin and 2-hydroxypropyl-gamma-cyclodextrin. The preferred amount of pharmaceutically acceptable beta- or gamma-cyclodextrin for use in the formulations is from 2 to 30% w/v, from 5 to 20% w/v, or from 15 to 30% w/v, and preferably is about 20% w/v or about 25% w/v.

Formulations including gedatolisib and methanesulphonic acid and/or ethanesulphonic acid are disclosed in PCT application publication WO2019/038657. The formulations include gedatolisib, or a methanesulphonate salt thereof, methanesulphonic acid, and water. The gedatolisib is present at a solution concentration of less than 35 mg/ml or up to 30 mg/ml (preferably from 6 to 30 mg/ml) and sufficient methanesulphonic acid is present to provide a clear solution. Another formulation disclosed is gedatolisib, or an ethanesulphonate salt thereof, ethanesulphonic acid and water. In embodiments, gedatolisib is present at a solution concentration of less than 35 mg/ml or up to 30 mg/ml (preferably from 6 to 30 mg/ml) and sufficient ethanesulphonic acid is present to provide a clear solution. In embodiments, gedatolisib is present at a concentration from about: 6 mg/mL, 8 mg/mL, 10 mg/mL, 12 mg/mL, 14 mg/mL, 16 mg/mL, 18 mg/mL, 20 mg/mL, 22 mg/mL, 24 mg/mL, 26 mg/mL, 28 mg/mL, or up to about 30 mg/mL.

The use of methanesulphonic acid and ethanesulphonic acid enables a solution concentration of up to 30 mg/ml of gedatolisib to be achieved for a pharmaceutical aqueous solution formulation that is suitable for intravenous or parenteral administration to a patient, i.e. a clear, essentially particle-free solution.

In the above aqueous formulations, a solution concentration of gedatolisib that is at least 6 mg/ml is desirable to allow dose administration to subjects using a single vial presentation of the commercial drug product. A lyophilized drug product (for reconstitution) containing less than 6 mg/ml drug product solution will require multiple vials to deliver the required therapeutic dose. A multiple vial approach to dose delivery is not desirable given current regulatory expectations for these product types.

Any of the above-mentioned formulations may be freeze-dried to provide a lyophilized solid composition, a bulking agent may be added to the formulation prior to the freeze-drying process commencing. A bulking agent may not be present if the formulation of the invention contains a pharmaceutically acceptable beta- or gamma-cyclodextrin. In embodiments, a bulking agent provides a freeze-dried solid with a non-collapsible, structural integrity that will allow rapid reconstitution on constitution of the aqueous formulation prior to administration, and it should also facilitate efficient lyophilization. Bulking agents are typically used when the total mass of solutes in the formulation is less than 2g/100 ml. Bulking agents may also be added to achieve isotonicity with blood. The bulking agent may be selected from a saccharide, sugar alcohol, amino acid or polymer, or be a mixture of two or more of any thereof. Preferably, the bulking agent is a sugar or sugar alcohol, or a mixture thereof. Preferably, the sugar is sucrose. Preferably, the sugar alcohol is mannitol. Constitution of the lyophilized solid composition may be achieved using an appropriate quantity of water and/or an aqueous solution of a suitable tonicity modifier in order to ensure that a clear solution is obtained.

Therapeutic agents containing at least one basic nitrogen atom (i.e., protonatable nitrogen-containing therapeutic agents), such as gedatolisib, represent an important group of therapeutic agents. However, nanoparticle formulations of this class of drugs are often hindered by undesirable properties, e.g., unfavorable burst release profiles and poor drug loading. PCT application publication WO2015/138835 discloses therapeutic nanoparticles of gedatolisib which have a controlled release rate of the therapeutic agent.

The therapeutic nanoparticles include gedatolisib (preferably in an amount of about 1 to 20 weight percent), a substantially hydrophobic acid, and a polymer selected from diblock poly (lactic) acid-poly (ethylene) glycol copolymer or a diblock poly (lactic acid-co-glycolic acid)-poly (ethylene) glycol copolymer, and combination thereof. The molar ratio of the substantially hydrophobic acid to the gedatolisib ranges from about 0.25:1 to about 2:1 and the pKa of the protonated gedatolisib is at least about 1.0 pKa units greater than the pKa of the hydrophobic acid. The hydrophobic acid and the gedatolisib form a hydrophobic ion pair in the therapeutic nanoparticle. Additionally, the nanoparticles can include a targeting ligand, which may increase target binding (cell binding/target uptake), making the nanoparticle target specific.

In general, a “nanoparticle” refers to any particle having a diameter of less than 1000 nm. Preferably, the therapeutic nanoparticles may have a diameter ranging from 60 to 120 nm. For example, the nanoparticle may have a diameter ranging from about 60 nm, about 70 nm, about 80 nm, about 90 nm, about 100 nm, or about 110 nm, up to about 70 nm, about 80 nm, about 90 nm, about 100 nm, about 110 nm, or about 120 nm.

As used herein, the “substantially hydrophobic acid” is an acid which has a pKa in water of about-1.0 to about 5.0. Preferably, the substantially hydrophobic acid has a pKa in water of about 2.0 to about 5.0. Exemplary substantially hydrophobic acids include, but are not limited to, fatty acids. For example, the fatty acid may be a saturated fatty acid, including, but not limited to, caproic acid, enanthic acid, caprylic acid, pelargonic acid, capric acid, undecanoic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, or combinations thereof. Additionally, the fatty acid may be a omega-3 fatty acid, including, but not limited to, hexadecatrienoic acid, alpha-linolenic acid, stearidonic acid, eicosatrienoic acid, cicosatetraenoic acid, eicosapentaenoic acid, heneicosapentaenoic acid, docosapentaenoic acid, docosahexaenoic acid, tetracosapentaenoic acid, tetracosahexaenoic acid, or combinations thereof. The fatty acid may also be an omega-6 fatty acid, including, but not limited to, linoleic acid, gamma-linolenic acid, cicosadienoic acid, dihomo-gamma-linolenic acid, arachidonic acid, docosadienoic acid, adrenic acid, docosapentaenoic acid, tetracosatetraenoic acid, tetracosapentaenoic acid, or combinations thereof. The fatty acid may also be an omega-9 fatty acid, including, but not limited to, oleic acid, cicosenoic acid, mead acid, erucic acid, nervonic acid, or combinations thereof. The fatty acid may also be a polyunsaturated fatty acid, including, but not limited, rumenic acid, a-calendic acid, β-calendic acid, jacaric acid, a-eleostearic acid, β-cleostearic acid, catalpic acid, punicic acid, rumelenic acid, a-parinaric acid, β-parinaric acid, bosseopentacnoic acid, pinolenic acid, podocarpic acid, or combinations thereof.

Alternatively, the hydrophobic acid can be a bile acid. For example, in some embodiments, the bile acid includes but is not limited to, chenodeoxycholic acid, ursodeoxycholic acid, deoxycholic acid, hycholic acid, beta-muricholic acid, cholic acid, lithocholic acid, an amino acid-conjugated bile acid, or combinations thereof.

Alternatively, the hydrophobic acid may include but is not limited to, dioctyl sulfosuccinic acid, 1-hydroxy-2-naphthoic acid, dodecylsulfuric acid, naphthalene-1,5-disulfonic acid, naphthalene-2-sulfonic acid, pamoic acid, undecanoic acid, or combinations thereof.

The nanoparticles may be combined with pharmaceutically acceptable carriers to form a pharmaceutical composition. As would be appreciated by one of skill in this art, the carriers may be chosen based on the route of administration, the location of the target issue, the time course of delivery of the drug, etc.

The pharmaceutical nanoparticle compositions can be administered to a patient or subject by any means known in the art including oral and parenteral routes. The nanoparticle compositions may be administered by injection (e.g., intravenous, subcutaneous or intramuscular, intraperitoneal injection), rectally, vaginally, topically (as by powders, creams, ointments, or drops), or by inhalation (as by sprays).

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Aqueous pharmaceutical formulations of gedatolisib, such as those described above, that are suitable for intravenous administration generally have a pH of from 3 to 9. However, lower pH values are tolerated in certain settings. The pH may range from about 3, 3.5, 4, 4.5, 5, 5.5, 6, 6.5, 7, 7.5, or 8 up to about 3.5, 4, 4.5, 5, 5.5, 6, 6.5, 7, 7.5, 8, 8.5, or 9. Preferably, the pH is from 3 to 8 or from 4 to 8.

In some embodiments, the gedatolisib used in the method of the present application can be formulated with one or more pharmaceutically acceptable excipients to form pharmaceutical compositions, such as intravenous formulations. Non-limiting examples of gedatolisib formulations suitable for intravenous delivery include those described above, such as those described in PCT publications WO 2016/097949, WO 2019/234632 and WO 2019/038657.

The pharmaceutical compositions used in the methods disclosed herein may be specially formulated in solid, semisolid, or liquid form, including those adapted for parenteral administration, for example, by intravenous, topical, intraocular injection, subcutaneous, intratumoral or intramuscular injection or infusion as, for example, a sterile solution or suspension.

A pharmaceutically acceptable excipient can be a pharmaceutically acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject, bulking agent, salt, surfactant and/or a preservative. Some examples of materials which can serve as pharmaceutically acceptable excipients include: sugars, such as lactose, glucose and sucrose and various polymers thereof; cyclodextrins, starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations.

A bulking agent is a compound that adds mass to a pharmaceutical formulation and contributes to the physical structure of the formulation in lyophilized form. Suitable bulking agents according to the present disclosure include mannitol, glycine, polyethylene glycol and sorbitol.

The use of a surfactant can reduce aggregation of a reconstituted protein and/or reduce the formation of particulates in the reconstituted formulation. The amount of surfactant added is such that it reduces aggregation of the reconstituted protein and minimizes the formation of particulates after reconstitution. Suitable surfactants according to the present disclosure include polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linolcyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linolcamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and polyethyl glycol, polypropyl glycol, and copolymers of ethylene and propylene glycol (e.g. Pluronics, PF68, etc.).

Preservatives may be used in formulations provided herein. Suitable preservatives for use in the formulation of the disclosure include octadecyldimethylbenzyl ammonium chloride, hexamethonium chloride, benzalkonium chloride (a mixture of alkylbenzyl-dimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. Other suitable excipients can be found in standard pharmaceutical texts, e.g. in “Remington's Pharmaceutical Sciences”, The Science and Practice of Pharmacy, 19th Ed. Mack Publishing Company, Easton, Pa., (1995).

In some embodiments, the gedatolisib used in the methods disclosed herein may be lyophilized and provided in a composition for reconstitution prior to administration.

### III. Hormone Therapy

The methods of the disclosure combine gedatolisib administration and hormone therapy in the treatment of hormonally driven disorders other than breast cancer. A wide variety of hormone therapies are well established in the art. In one embodiment, the hormone therapy is an anti-androgen therapy. Non-limiting exemplary anti-androgen therapies are described further below. In another embodiment, the hormone therapy is an anti-estrogen therapy. Non-limiting exemplary anti-estrogen therapies are described further below.

A suitable hormone therapy is selected for use in the disorder based on the hormone drivenness involved in the disorder, such as testosterone or estrogen/estradiol. For example, in treatment of prostate cancer, an anti-androgen therapy is used as the hormone therapy, whereas in endometrial disorders, an anti-estrogen therapy is used as the hormone therapy. Combinations of hormone therapies are also contemplated in the methods herein.

### Anti-Androgen Therapies

In embodiments, the hormone therapy comprises an anti-androgen therapy, which reduces the level of androgens (e.g., testosterone) in the subject. In embodiments, the anti-androgen therapy comprises administration of an androgen receptor signaling inhibitor (ARsi). As used herein, an “ARsi” is intended to encompass inhibitors (e.g., antagonists) of the androgen receptor that reduce signaling through the receptor by any of a variety of different mechanisms established in the art, including the following anti-androgen therapies.

In embodiments, anti-androgen therapies comprising use of an ARsi include administration of a non-steroidal androgen receptor inhibitor, administration of a steroidal anti-androgen, administration of an androgen synthesis inhibitor, administration of an androgen receptor pathway inhibitor, administration of an androgen receptor protein degrader, or administration of an N-terminal domain (NTD) androgen receptor inhibitor.

In embodiments, the hormone therapy comprises administering to the subject a non-steroidal androgen receptor inhibitor. Non-limiting examples of non-steroidal androgen receptor inhibitors include darolutamide, apalutamide, bicalutamide, enzalutamide, flutamide, nilutamide and topilutamide. In embodiments, the non-steroidal androgen receptor inhibitor is used at its recommended dosage and recommended treatment schedule.

In an embodiment, the ARsi is darolutamide (NUBEQA®), which has received approval for treatment of patients with nonmetastatic CRPC and in metastatic hormone-sensitive prostate cancer. In embodiments, darolutamide is used at its recommended dosage of 600 mg twice daily, administered orally, for a total daily dosage of 1200 mg.

In embodiments, the hormone therapy comprises administering to the subject a steroidal anti-androgen. In embodiments, the steroidal anti-androgen is a progesterone derivative, a testosterone derivative, a spirolactone derivative or a cortisol derivative.

In embodiments, the steroidal anti-androgen is a progesterone derivative, non-limiting examples of which include 11α-hydroxyprogesterone, Chlormadinone acetate, Clometerone, Cyproterone, Cyproterone acetate, Edogestrone, Medrogestone, Megestrol acetate, Nomegestrol acetate and Osatcrone acetate. In embodiments, the progesterone derivative is used at its recommended dosage and recommended treatment schedule.

In embodiments, the steroidal anti-androgen is a testosterone derivative, non-limiting examples of which include Benorterone. 6 alpha-bromo-17 beta-hydroxy-17 alpha-methyl-4-oxa-5 alpha-androstan-3-one (BOMT), Delanterone, Dienogest, Epitestosterone, Galeterone, Mctogest, Oxendolone, Rosterolone, Topterone, Trimethyltrienolone and Zanoterone. In embodiments, the testosterone derivative is used at its recommended dosage and recommended treatment schedule.

In embodiments, the steroidal anti-androgen is a spirolactone derivative, non-limiting examples of which include 3-oxopregn-4-ene-21,17alpha-carbolactone (SC-5233), (8R,9S, 10R, 13S, 14S, 17R)-13-methylspiro[1,2,6,7,8,9,10,11,12,14,15,16-dodecahydrocyclopenta[a]phenanthrene-17,5′-oxolane]-2′,3-dione (SC-8109), Canrenone, Dicirenone, Drospirenone, Mespirenone, Mexrenone, Prorenone, Spironolactone, Spirorenone and Spiroxasone. In embodiments, the spirolactone derivative is used at its recommended dosage.

In embodiments, the steroidal anti-androgen is a cortisol derivative, non-limiting examples of which include 9,11-Dehydrocortexolone 17α-butyrate and Clascoterone. In embodiments, the cortisol derivative is used at its recommended dosage and recommended treatment schedule.

In embodiments, the hormone therapy comprises administering to the subject an androgen synthesis inhibitor, non-limiting examples of which include Cyp17A antagonists for non-limiting examples Abiraterone, Abiraterone acetate, and 5α-reductase antagonists for non-limiting examples finasteride, dutasteride and leuprolide. In embodiments, the androgen synthesis inhibitor is used at its recommended dosage and recommended treatment schedule.

In embodiments, the hormone therapy comprises administering to the subject an androgen receptor pathway inhibitor, a non-limiting example of which is abiraterone acetate. In embodiments, the androgen receptor pathway inhibitor is used at its recommended dosage. In embodiments, the hormone therapy comprises administering to the subject an androgen receptor protein degrader inhibitor, a non-limiting example of which is ARV-766. In embodiments, the androgen receptor protein degrader inhibitor is used at its recommended dosage and recommended treatment schedule.

In embodiments, the hormone therapy comprises administering to the subject an antagonist that targets the N-terminal domain (NTD) of the androgen receptor (AR), a non-limiting example of which is masofaniten. In embodiments, the antagonist that targets the NTD of the AR is used at its recommended dosage and recommended treatment schedule.

In embodiments, the hormone therapy comprises administering to the subject a non-steroidal, selective inhibitor of steroid biosynthesis, a non-limiting example of which is MK-5684. In embodiments, the non-steroidal, selective inhibitor of steroid biosynthesis is used at its recommended dosage and recommended treatment schedule.

### Anti-Estrogen Therapies

In embodiments, the hormone therapy comprises an anti-estrogen therapy, which reduces the level of estrogens in the subject. Anti-estrogen therapies include administration of a selective estrogen receptor modulator (SERM), administration of a selective estrogen receptor degrader (SERD) and administration of an aromatase inhibitor.

In embodiments, the hormone therapy comprises administering to the subject a selective estrogen receptor modulator (SERM). Non-limiting examples of SERMs include tamoxifen, raloxifene, toremifene, ospemifene, bazedoxifene, ormeloxifene, lasofoxifene, cyclofenil, clomifene, broparestrol and anordin. In embodiments, the SERM is used at its recommended dosage and recommended treatment schedule.

In embodiments, the hormone therapy comprises administration of tamoxifen. Tamoxifen citrate is available as an orally administered tablet (e.g., Nolvadex™, Soltamox™). Tamoxifen typically is used in a dosage range of 10-40 mg daily. In embodiments, tamoxifen is administered at a dosage of 10 mg daily, or a dosage of 20 mg daily, or at a dosage of 30 mg daily or at a dosage of 40 mg daily.

In embodiments, the hormone therapy comprises administering to the subject a selective estrogen receptor degrader (SERD). Non-limiting examples of SERDs include fulvestrant, elacestrant, amcenestrant, camizestrant, giredestrant, imlunestrant, vepdegestrant, palazestrant, LSZ102, rintodestrant, SHR9549, H3B-5942, ZN-c5, and brilanestrant. In embodiments, the SERD is used at its recommended dosage and recommended treatment schedule.

In embodiments, the hormone therapy comprises administration of fulvestrant. Fulvestrant (Faslodex™, AstraZeneca, Cambridge, UK) is available as an injection for intramuscular administration, supplied as a 250 mg/5 mL vial. See Faslodex™ Prescribing Information. The recommended dosing of fulvestrant is 500 mg intramuscularly into the buttocks (gluteal area) slowly as two 5 mL injections, on Days 1, 15, 29, and once monthly thereafter. See id. For patients with moderate hepatic impairment the recommended dose is 250 mg administered intramuscularly as one 5 mL injection on Days 1, 15, 29, and once monthly thereafter. See id.

In embodiments, the hormone therapy comprises administering to the subject an aromatase inhibitor. Non-limiting examples of aromatase inhibitors include letrozole, anastrozole and exemestane. In embodiments, the aromatase inhibitor is used at its recommended dosage and recommended treatment schedule.

In embodiments, the hormone therapy comprises administration of letrozole. Letrozole (Femara™; Novartis) is available as an orally administered tablet. In embodiments, letrozole is administered orally at a dosage of 2.5 mg once a day (daily).

Additional estrogen receptor modulators are known in the art. For example, ARN-810 (GDC-0810, Seragon Pharmaceuticals, Genentech Inc.) is a small molecule, nonsteroidal, selective ER modulator that antagonizes the effects of estrogens and induces ER degradation via proteasome. ARN-810 is in clinical trials as an orally-delivered therapy to treat advanced metastatic ER-α positive (ER+) breast cancer.

PCT application publication WO2013/090836 discloses fluorinated estrogen receptor modulators and uses thereof.

PCT application publication WO2014/205136 discloses azetidine estrogen receptor modulators and uses thereof.

U.S. patent application publication no. 2003/0130274 discloses 2-phenyl-1-[4-(2-aminoethoxy)benzyl]-indoles as estrogenic agents.

### IV. Additional Therapies

In embodiments, the methods of the disclosure combining gedatolisib administration and hormone therapy further comprise treating the subject with one or more additional therapies suitable for use with the hormonally driven disorder under treatment. Non-limiting examples of additional therapies include administering to the subject one or more chemotherapeutic drugs, administering to the subject one or more immune checkpoint inhibitors and performing on the subject one or more surgical treatments, such as a surgery that alters hormones (e.g., testosterone) in the subject. Standard surgical interventions used in the treatment of hormonally driven disorders are well-established in the art, non-limiting examples of which include radical prostatectomy, orchidectomy, total hysterectomy, partial hysterectomy, radical hysterectomy, removal of ovaries, removal of fallopian tubes, removal of cervix, and laparoscopy for endometriosis.

### Chemotherapeutic Agents

In embodiments, gedatolisib administration and hormone therapy are further combined with treatment of the subject with one or more chemotherapeutic agents.

As used herein the term “chemotherapy” or “chemotherapeutic agent” refers to treatment with a cytostatic or cytotoxic agent (i.e., a compound) to reduce or eliminate the growth or proliferation of undesirable cells, for example cancer cells. Thus, as used herein, “chemotherapy” or “chemotherapeutic agent” refers to a cytotoxic or cytostatic agent used to treat a proliferative disorder, for example cancer.

Exemplary cytotoxic chemotherapy pharmaceutical compounds include, but are not limited to, a cyclophosphamide, an ifosamide, a methotrexate, a substituted nucleotide, a substituted nucleoside, fluorouracil, a mitomycin, adriamycin, vincristine, vindesine, taxol, cisplatin, carboplatin, etoposide, paclitaxel, docetaxel, or a combination thereof.

In embodiments, the chemotherapeutic agent is used at its recommended dosage and recommended treatment schedule.

### Targeted Therapy Combinations

In embodiments, the targeted therapeutic agent is an inhibitor of a signaling molecule or surface receptor, such as a CDK 4/6 inhibitor or a Receptor Tyrosine Kinase (RTK) inhibitor, such as an EGFR inhibitor or a therapeutic that targets an immune checkpoint regulator or a therapeutic that targets an epigenetic regulatory process.

### Therapies Targeting Oncogenic Proteins or Targeting Translation of Oncogenic or Stress Related Proteins

In embodiments, a targeted therapy used in combination in a method of the disclosure comprises a therapy that targets oncogenic proteins or that targets translation of oncogenic or stress-related proteins. Tumor cells are especially plastic and adept at circumventing stressful environments for example nutrient starvation, low pH, and hypoxia. Targeted therapies have been developed to reduce these opportunities employed by tumor cells to remain viable and proliferative by targeting specific oncogenic drivers. Oncogenic proteins are well known to oncologists and include proteins for non-limiting examples Ras, Raf, Myc, proteins in the eukaryotic translation initiation complex (e.g. eIF4A, eIF4E, and members of the cIF4 complex) responsible for translating stress related proteins via 5′ cap independent mechanisms (see e.g., Sanchez-Vega et al. (2018) Cell 173:321-337.e10; Senga and Grose (2021) Open Biol. 11:200358; and Hanahan and Weinberg (2011) Cell 144:646-674). Non-limiting examples of therapies targeting oncogenic proteins include: Zotatifin (cIF4A inhibitor); KSI3716 and ML327 and WBC100 (Myc inhibitors); sotorasib (Lumakras, KRas inhibitor), MRTX 849 (adagrasib, KRas inhibitor), Vemurafenib and dabrafenib and encorafenib (BRaf inhibitors). In embodiments, the therapy targeting an oncogenic protein is used at its recommended dosage and recommended treatment schedule.

### CDK 4/6 Inhibitors

In embodiments, the additional targeted therapeutic treatment comprises administration of a CDK 4/6 inhibitor, such as palbociclib. As used herein, the term “CDK 4/6 inhibitor” includes compounds that inhibit CDK4 activity, CDK6 activity, or both CDK4 and CDK6 activity.

In embodiments, administration of palbociclib is synchronized with administration of gedatolisib (e.g., both on a treatment schedule of 3 weeks on/1 week off; see section V).

The regulation of the cell cycle is governed and controlled by specific proteins, which are activated and deactivated mainly through phosphorylation/dephosphorylation processes in a precisely timed manner. The key proteins that coordinate the initiation, progression, and completion of cell-cycle program are cyclin dependent kinases (CDKs). Cyclin-dependent kinases belong to the serine-threonine protein kinase family. They are heterodimeric complexes composed of a catalytic kinase subunit and a regulatory cyclin subunit. CDK activity is controlled by association with their corresponding regulatory subunits (cyclins) and CDK inhibitor proteins (Cip & Kip proteins, INK4s), by their phosphorylation state, and by ubiquitin-mediated proteolytic degradation.

There are four CDKs that are significantly involved in cellular proliferation: CDK1, which predominantly regulates the transition from G2 to M phase, and CDK2, CDK4, and CDK6, which regulate the transition from G1 to S phase. In early to mid G1 phase, when the cell is driven to mitogenic stimuli, activation of CDK4-cyclin D and CDK6-cyclin D induces phosphorylation of the retinoblastoma protein (pRb). Phosphorylation of pRb releases the transcription factor E2F, which enters the nucleus to activate transcription of other cyclins which promote further progression of the cell cycle. The link between CDK and PI3K signaling has also been described in the art (for reviews, see e.g., Clark et al. (2021) Clin. Cancer Res. 27:371-373; Pandey et al. (2019) Int. J. Cancer 145:1179-1188; Portman et al. (2019) Endocr. Relat. Cancer 26: R15-R30; and Presti et al. (2019) Cancers (Basel) 11:1242).

CDK4/6 inhibitors, including palbociclib, abemaciclib, and ribociclib, have become the standard of care for treatment of advanced breast cancers, and are now being explored in early breast cancers. The active CyclinD/CDK4/6 complex phosphorylates RB, causing its dissociation from E2F, which frees E2F to target transcription of genes driving cell cycle progression. CDK4/6 inhibitors prevent the CyclinD/CDK complex from phosphorylating RB and therefore blocking cell cycle progression. mTORC1 also activates CyclinD/CDK to drive cell cycle. Cancers eventually develop resistance to CDK4/6 inhibitors by several mechanisms including RB dysfunction, CyclinE (CCNE1) upregulation, and upregulation of PI3K/mTOR signaling. Similarly, insensitivity to PI3Ki was linked to persistent RB phosphorylation and could be effectively overcome by adding a CDK inhibitor (Vora et al. (2014) Cancer Cell 26:136-149). The combination of CDKi and PAMi is a promising therapeutic strategy explored in several nonclinical and clinical studies.

A number of CDK 4/6 inhibitors have been identified, including specific pyrido[2,3-d]pyrimidines, 2-anilinopyrimidines, diaryl ureas, benzoyl-2,4-diaminothiazoles, indolo[6,7-a]pyrrolo[3,4-c]carbazoles, and oxindoles. For example, WO 03/062236 identifies a series of 2-(pyridin-2-ylamino-pyrido[2,3]pyrimidin-7-ones for the treatment of Rb positive cancers that show selectivity for CDK4/6, including 6-acetyl-8-cyclopentyl-5-methyl-2-(5-piperazin-1-yl-pyridin-2-ylammino)-8H-pyrido-[2,3-d]-pyrimidin-7-one (PD0332991). Tate, et al. describe the antitumor activity of the CDK4/6 inhibitor abemaciclib (LY2835219) (“Semi-Mechanistic Pharmacokinetic/Pharmacodynamic Modeling of the Antitumor Activity of LY2835219, a New Cyclin-Dependent Kinase 4/6 Inhibitor, in Mice Bearing Human Tumor Xenografts”, Clin Cancer Res (Jul. 15, 2014) 20; 3763). Rader, et al. describe the reduced proliferation in neuroblastoma-derived cell lines using the CDK4/6 inhibitor ribociclib (LEE011) (“Dual CDK4/CDK6 Inhibition Induces Cell Cycle Arrest and Senescence in Neuroblastoma”, Clin Cancer Res (Nov. 15, 2013) 19 (22): 6173-82). VanderWel et al. describe an iodine-containing pyrido[2,3-d]pyrimidine-7-one (CKIA) as a potent and selective CDK4 inhibitor (see VanderWel et al., J. Med. Chem. 48 (2005) 2371-2387). WO 99/15500 filed by Glaxo Group Ltd discloses protein kinase and serine/threonine kinase inhibitors. WO 2010/020675 filed by Novartis AG describes pyrrolopyrimidine compounds as CDK inhibitors. WO 2011/101409 also filed by Novartis describes pyrrolopyrimidines with CDK 4/6 inhibitory activity. WO 2005/052147 filed by Novartis and WO 2006/074985 filed by Janssen Pharma disclose additional CDK4 inhibitors. WO 2012/061156 filed by Tavares and assigned to G1 Therapeutics describes CDK inhibitors. WO 2013/148748 filed by Francis Tavares and assigned to G1 Therapeutics describes Lactam Kinase Inhibitors.

Selective CDK4/6 inhibitors are generally designed to target CDK4/6-replication dependent cancers. For example, Michaud et al., reported that the CDK4/6 inhibitor PD-0332991 was inactive against Rb-negative tumors. (Michaud et al., Pharmacologic Inhibition of Cyclin-Dependent Kinase 4 and 6 Arrests the Growth of Glioblastoma Multiform Intracranial Xenografts. Cancer Res. 70:3228-3238 (2010)).

In some embodiments, the CDK 4/6 inhibitor is selected from the group consisting of palbociclib, ribociclib, abemaciclib, trilaciclib, dalpiciclib, riviciclib, and combinations thereof. In embodiments, the CDK 4/6 inhibitor is palbociclib. In embodiments, the CDK 4/6 inhibitor is ribociclib. In embodiments, the CDK 4/6 inhibitor is abemaciclib. In embodiments, the CDK 4/6 inhibitor is trilaciclib. In embodiments, the CDK 4/6 inhibitor is dalpiciclib. In embodiments, the CDK 4/6 inhibitor is riviciclib.

The CDK 4/6 inhibitor may be administered using the methods as known in the art. In some embodiment, the CDK 4/6 inhibitor is palbociclib. Palbociclib (Ibrance™, Pfizer, New York, NY) is available in 125 mg, 100 mg, and 75 mg tablets and capsules. See Ibrance™ Prescribing Information. The recommended dose of palbociclib is 125 mg taken orally once daily for 21 consecutive days followed by 7 days off treatment to comprise a complete cycle of 28 days. Id. This treatment cycle may be modified based on the results of treatment and the tolerance of the patient. Id. For example, if patients experience neutropenia the administration of the palbociclib can be reduced to 100 mg, or 75 mg once daily for 21 consecutive days followed by 7 days off treatment. Id.

Ribociclib (Kisqali™, Novartis, Switzerland) is available in 200 mg tablets. See Kisqali™ Prescribing Information. The recommended dose of ribociclib is 600 mg (three 200 mg tables) taken orally once daily for 21 consecutive days followed by 7 days off treatment to comprise a complete cycle of 28 days. Id. This treatment cycle may be modified based on the results of treatment and the tolerance of the patient. Id. For example, if patients experience negative side effects administration of the ribociclib can be reduced to 400 mg, or 200 mg once daily for 21 consecutive days followed by 7 days off treatment. Id.

Abemaciclib (Verzenio™, Eli Lilly, Indianapolis, IN) is available in 200 mg, 150 mg, 100 mg, and 50 mg tablets. See Verzenio™ Prescribing Information. The recommended dose of abemaciclib when administered in combination with fulvestrant is 150 mg twice daily. Id. The recommended dose of abemaciclib when administered without fulvestrant is 200 mg twice daily. Id. If dose reduction is necessary, it is recommended to reduce the abemaciclib dose by 50 mg at a time. Id.

Trilaciclib (Cosela™, G1 Therapeutics, Inc., NC) is available in a 300 mg lyophilized powder in a single-dose vial. See Cosela™ Prescribing Information. The recommended dose of trilaciclib is 240 mg/m2 per dose administered as an intravenous (IV) infusion. Id. The trilaciclib should be reconstituted with 19.5 mL of 0.9% Sodium Chloride Injection or 5% Dextrose Injection, USP, to obtain a concentration of 15 mg/mL. Id. Trilaciclib is generally administered as 30-minute intravenous infusion which must be completed within 4 hours prior to the start of chemotherapy on each day chemotherapy is administered. Id.

Dalpiciclib (SHR6390, Jiangsu Hengrui Medicine Co.) is in clinical trials and has been dosed 150 mg, orally once daily on Day 1 to Day 21 of every 28-day cycle, followed by 7 days off treatment. See NCT04236310, Jan. 17, 2020. Dalpiciclib is currently being investigated in in combination with letrozole or anastrozole or fulvestrant in patients with HR-positive and HER2-negative advanced breast cancer.

Riviciclib (P276-00, Piramal Enterprises Ltd., Mumbai, IN) is in clinical trials and has been administered as an intravenous infusion at a concentration of 185 mg/m2 in 200 ml of 5% dextrose over 30 min per day from days 1 to 5 of a 21-day cycle. See NCT00898287, Jan. 20, 2012. Riviciclib has been investigated in in combination with gemcitabine and carboplatin in patients with metastatic triple negative breast cancer.

Atirmociclib (PF-07220060, Pfizer Inc.) is a CDK4 inhibitor and is clinical trials. Atirmociclib is currently being investigated in in combination with letrozole or fulvestrant in patients with HR-positive and HER2-negative advanced breast cancer.

In embodiments, the CDK 4/6 inhibitor is used at its recommended dosage and recommended treatment schedule.

### RTK Inhibitors

In embodiments, the additional targeted therapeutic treatment comprises administration of a receptor tyrosine kinase (RTK) inhibitor. In an embodiment, the RTK inhibitor is a small molecule inhibitor. In an embodiment, the RTK inhibitor is a therapeutic antibody, such as a monoclonal antibody targeting a receptor. In embodiments, the therapeutic antibody is conjugated to a therapeutic agent, such as a toxin (antibody-drug conjugate). A wide variety of RTK inhibitors and their use in cancer therapy have been described in the art, reviewed for example in Roskoski (2024) Pharmacol. Res. 200:107059.

In some embodiments, the RTK inhibitor is selected from the group consisting of: erlotinib, gefitinib, lapatinib, vandetanib, afatinib, panitumumab, cetuximab, brigatinib, icotinib, osimertinib, neratinib, zalutumumab, nimotuzumab, matuzumab, pertuzumab, trastuzumab, dacomitinib, acomitinib, BIBW2992, tesevatinib, amuvatinib, necitumumab, REGN955, MM-151, nazartinib, ASP8273, olmutinib, TDM1, MEDI4276, ZW25, ZW33, tucatinib, rocilctinib, ibrutinib, DS-8201, TAS07828, XMT-1522, TAK-788, Sym013, LIM716, scribantumab, AMG888, lumretuzumab, PF-06804103, ARX788, poziotinib, pyrotinib, duligotuzuman, MCLA-128, MM-111, cabozantinib, tivantinib, crizotinib (PF-2341066), tepotinib, capmatinib, savolitinib, K252a, SU11274, PHA-665752, ARQ197, forctinib, SGX523, MP470, AV229, AMG102, CGEN241, DN30, OA5D5, rilotumumab, onartuzumab, SAR125844, cmbetuzumab, ABBV-399, sym015, ficlatuzumab, merestinib, JNJ-61186372, altiratinib, Indo5, BMS-754807, BMS-777607, glesatinib, CEP-751, ANA-12, cyclotraxin B, gossypetin, entrectinib, larotrectinib, LOXO-101, dovotinib, lenvatinib, ponatinib, regorafenib, lucitanib, cediranib, intedanib, brivanib, PD173074, AZD4547, BGJ398, JNJ42756493, GP369, BAY1187982, MFGR1877S, FP1039, pazopanib, erdafitinib, Debio-1347, B-701, fisogatinib, FIIN-2, FIIN-3, BLU9931, LY2874455, LY3076226, sunitinib, AG538, AG1024, NVP-AEW541, figitumumab, linsitinib, dalotuzumab, MEDI-573, teprotumumab, ganitumab, ceritinib, MM-141, cofctuzumab pelidotin (PF-06647020), dasatinib, nilotinib, NVP-BHG712, sitravatinib, ALW-II-41-27, JI-101, 123C4, sorafenib, apatinib, AST487, alectinib, dovitinib, crizotinib, lorlatinib, TPX-0005, DS-6051b, imatinib, linifanib, KTN0182A, gilteritinib, quizartinib, midostaurin, lestaurtinib, ripretinib, masitinib, avapritinib, pexidartinib, telatinib, motesanib, PLX7486, ARRY386, JNJ-40346527, BLZ945, cmactuzumab, AMG820, IMC-CS4, cabiralizumab, CHMFL-KIT-033, SU14813, Ki20227, OSI-930, flumatinib, toceranib, AZD3229, AC710, AZD2932, ICK03, PLX647, c-Kit-IN-3, vatalanib, bevacizumab, rebastinib, BAY-826, bemcentinib, R428 (BGB324), YW327.6S2, GL2I.T, TP-0903, LY2801653, bosutinib, MGCD265, ASP2215, SGI-7079, BGB324, HuMax-AXL-ADC, and UC-961. In some embodiments, the RTK inhibitor and RTK targeted therapeutic are the same compound. In other embodiments, the RTK inhibitor and RTK targeted therapeutic are different compounds. In embodiments, the RTK inhibitor is used at its recommended dosage and recommended treatment schedule.

### Immune Checkpoint Inhibitors

In embodiments, the additional targeted therapeutic treatment comprises administration of an immune checkpoint inhibitor (ICI). In embodiments, the ICI is a biologic therapeutic. In embodiments, the ICI is a small molecule. For example, the ICI can be a monoclonal antibody, a humanized antibody, a fully human antibody, a fusion protein or a combination thereof. The ICI typically is used as the recommended dosage and treatment schedule, such as the dosage and treatment schedule set forth on the FDA-approved label for the ICI.

The checkpoint inhibitor may inhibit a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands and combination thereof. The checkpoint inhibitor can interact with a ligand of a checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof.

In embodiments, the ICI is a PD-1 inhibitor. Non-limiting examples of PD-1 inhibitors include pembrolizumab (Keytruda™), nivolumab (Opdivo™) and cemiplimab (Libtayo™).

In embodiments, the ICI is a PD-L1 inhibitor. Non-limiting examples of PD-L1 inhibitors include atezolizumab (Tecentriq™), avelumab (Bavencio™) and druvalumab (Imfinzi™).

In embodiments, the ICI is a CTLA4 inhibitor. Non-limiting examples of CTLA4 inhibitors include ipilimumab (Yervoy™) and tremeliumumab (Imjuno™).

In embodiments, the immune checkpoint inhibitor is used at its recommended dosage and recommended treatment schedule.

### Epigenetic-Targeted Therapies

In other embodiments, an additional therapy used in a method of the disclosure targets an epigenetic regulator(s) involved in an epigenetic process(es), referred to herein as an epigenetic-targeted therapy. Epigenetic factors are those beyond the genetic code and epigenetic processes are involved in regulating the timing and/or amount of gene transcription. Examples of epigenetic processes involving heritable but reversible changes that regulate gene activity beyond the underlying genetic sequence include DNA modifications and histone modifications. Dysregulation of epigenetic processes has been implicated in the development and/or progression of cancer. Therapies targeting various molecules involved in epigenetic processes have been described in the art (reviewed in, for example Cheng et al. (2019) Signal Transduction and Targeted Therapy 4:62).

Thus, in embodiments, a method of the disclosure that combines gedatolisib administration with hormone therapy further comprises an epigenetic-targeted therapy. As used herein an “epigenetic-targeted therapy” refers to a therapy (e.g., administration of a drug) that targets one or more epigenetic regulators, such as administration of a drug that specifically inhibits the epigenetic regulator. An “epigenetic regulator” refers to a molecule (e.g., enzyme) involved in an epigenetic process, such as DNA modification or histone modification. Non-limiting examples of epigenetic modifications of DNA and histones include methylation and acetylation.

In embodiments, the epigenetic-targeted therapy comprises administration of an inhibitor of an epigenetic regulator, also referred to herein as an epigenetic inhibitor. In an embodiment, the epigenetic inhibitor is a small molecule inhibitor. In an embodiment, the epigenetic inhibitor is a therapeutic antibody, such as a monoclonal antibody that identifies cancer cell surface markers to provide entry of an epigenetic inhibitor. In embodiments, the therapeutic antibody is conjugated to a therapeutic agent, such as a toxin (antibody-drug conjugate, also known in the art as ADC drugs).

Non-limiting examples of epigenetic-targeted therapies that target an epigenetic regulator include the following:

### Histone Deacetylase Inhibitors (HDACIs)

In embodiments, the epigenetic therapy comprises administering to the subject a histone deacetylase inhibitor (HDACI). These drugs prevent the removal of acetyl groups from histone proteins, leading to increased gene transcription. HDACIs can inhibit the proliferation of tumor cells by, for example, inducing apoptosis, cell cycle arrest or differentiation of the cells. The anti-tumor effects of HDACIs can result from, for example, inducing changes in the expression of oncogenes and/or reducing expression of tumor suppressor genes through modulating the acetylation/deacetylation of histones and/or non-histone proteins such as transcription factors. Histone deacetylases and their targeting pharmacologically are reviewed in, for example, Milazzo et al. (2020) Genes 11:556. A number of HDACIs have been FDA-approved for use cancer. Non-limiting examples of HDACi that can be used as a combination agent in a method of the disclosure include Vorinostat (Zolinza™), Romidepsin (Istodax™), Belinostat (Beleodaq™) and Panobinostat (LBH-589). In embodiments, the DHACI is used at its recommended dosage and recommended treatment schedule.

### Lysine (K) Acetyltransferase 6 (KAT6) Inhibitors

In embodiments, the epigenetic therapy comprises administering to the subject a Lysine (K) acetyltransferase 6 (KAT6) inhibitor. KAT6 enzymes, including KAT6A and KAT6B, regulate gene transcription via acetylation of histone H3K23. Thus, KAT 6 is an epigenetic regulatory enzyme impacting histone acetylation and subsequent gene-specific changes. Such changes can alter transcription of oncogenes and/or tumor suppressor genes such that KAT6 can function as a cancer driver. For example, dysregulation of KAT6 activity can drive lineage-specific gene expression in cancer. KAT6 inhibitors are reviewed in, for example, White et al. (2024) Trends Pharm. Sci. 45:243-254. KAT6 inhibitors have been described in the art for use in cancers (see e.g., Sharma et al. (2021) Cancer Res. 81: Suppl. Abstr. 1130; Sharma et al. (2023) Cell Chem. Biol. 30:1191-1210; Sommerhalder et al. (2023) J. Clin. Oncol. 41: Suppl. Abstr. 1054; Mukohara et al. (2024) Nature Med. 30:2242-2250; Cai et al. (2024) Cancer Res. 84: Suppl. Abstr. P02-05-08). Non-limiting examples of KAT6 inhibitors that can be used as a combination agent in a method of the disclosure include PF-07248144 (Pfizer), OP-3136 (Olema Pharmaceuticals), ISM5043 (InSilico Medicine) CTx-648 (PF-9363; Pfizer) and KAT6-IN-1 (CAS #2569008-99-5). In embodiments, the KAT6 inhibitor is used at its recommended dosage and recommended treatment schedule.

### DNA Methyltransferase Inhibitors (DNMTi)

In another embodiment, the epigenetic therapy comprises administering to the subject a DNA methyltransferase inhibitor (DNMTi). DNA methyltransferases are enzymes that add a methyl group to DNA. Inhibitors of these enzymes inhibit such methylation, leading to gene activation. Hypomethylation of DNA can lead to transcription of previously silenced genes, such as oncogenes. DNMT inhibitors and their use in cancer therapy are reviewed in, for example, Dan et al. (2019) OncoTargets and Therap. 12:10903-10916 and Hu et al. (2021) Clinical Epigenetics 13:166. Non-limiting examples of DNMTi approved for use in the treatment of cancer include Azacitidine (Vidaza™) and Decitabine (Dacogen™). Additional non-limiting examples of DNMTi under development include Zebularine, 4′-thio-2′-deoxycytidine (TdCyd), SGI-1027, MG98, N-Phthaloyl-L-tryptophan (RG108) and Nanaomycin A. In embodiments, the DNMTi is used at its recommended dosage and recommended treatment schedule.

### Histone Methyltransferase (HMT) Targeting Small Molecule

In another embodiment, the epigenetic therapy comprises administering to the subject a small molecule targeting a histone methyltransferase (HMT), such as an HMT inhibitor. HMTs are enzymes that modify histones by catalyzing the transfer of 1-3 methyl groups to lysine or arginine residues of histone proteins, referred to as histone-lysine N-methyltransferases and histone-arginine N-methyltransferases, respectively. Use of HMT inhibitors in cancer therapy is reviewed in, for example, Marzochi et al. (2023) Eur. J. Pharmacol. 944:175590 and Rugo et al. (2020) Adv. Therap. 37:3059-3082. Non-limiting examples of HMT inhibitors include Tazemetostat (Tazverik™), an EZH2 inhibitor (inhibitor of Enhancer of Zeste Homolog 2, a histone-lysine N-methyltransferase) that has been FDA-approved in cancer treatment, as well as mevrometostat, valemetostat, HH2853, SHR2554, XNW5004, BR1733, AXT-1003 tulmimetostat, HM97662, and DW91170. In embodiments, the HMT targeting small molecule (e.g., HMT inhibitor) is used at its recommended dosage and recommended treatment schedule.

### Bromodomain Targeting Small Molecules

In another embodiment, the epigenetic therapy comprises administering to the subject a small molecule targeting a bromodomain of a Bromodomain and Extra-Terminal motif (BET) protein. BET proteins include BRD2, BRD3, BRD4 and BRDT. Bromodomain targeting small molecules, such as BET inhibitors, prevent protein-protein interactions between BET proteins, acetylated histones and transcription factors. BET inhibitors and their use in cancer therapy are reviewed in, for example, To et al. (2023) Molecules 28:3043 and Wang et al. (2023) Signal Transduction and Targeted Therapy 8:420. Non-limiting examples of BET inhibitors include Pelabresib (CPI-06 10), Trotabresib, ZEN-3694, BMS-986158, NUV-868, PLX51107 and ABBV-744. In embodiments, the bromodomain targeting small molecule (e.g., BET inhibitor) is used at its recommended dosage and recommended treatment schedule.

### Histone Lysine Demethylase Targeting Small Molecules

Demethylases are enzymes that remove methyl groups from molecules such as nucleic acids and proteins, including DNA and histones. Thus, demethylases are epigenetic regulators that modulate transcriptional regulation by modulating methylation levels on histones and DNA. Histone lysine demethylases are classified according to their domains and lysine substate specificities, non-limiting examples of which include Lysine-specific histone demethylase 1 (LSD1) as well as the KDM1, KDM2, KDM3, KDM4, KDM5 and KDM6 classes of enzymes. Histone lysine demethylase inhibitors and their use in cancer therapy are reviewed in, for example, Jambhekar et al. (2017) Cold Spring Harbor Perspect. Med. 7: a026484 and Perillo et al. (2020) Exp. Mol. Med. 52:1936-1947. Non-limiting examples of histone lysine demethylase inhibitors include tranylcypromine, iadademstat (ORY-1001), bomedemstat (IMG-7289), GSK-2879552, INCB059872, JBI-802, Phenelzinc., pulrodemstat (CC-90011) and seclidemstat (SP-2577). In embodiments, the histone lysine demethylase inhibitor (e.g., LSD1 inhibitor) is used at its recommended dosage and recommended treatment schedule.

Additional non-limiting examples of epigenetic regulator that can be targeted in an epigenetic-targeted therapy (e.g., using an inhibitor of the regulator) include methyl-CpG recognition proteins, acetyl-lysine recognition proteins and methyl-histone recognition proteins. Examples of such epigenetic-targeted therapies are known in the art and described in, for example, Cheng et al. (2019) Signal Transduction and Targeted Therapy 4:62). Additionally, mRNA based epigenetic targeted therapies, for example OTX-2002, are included in examples of targeting epigenetic regulators at either the DNA or protein level described in for example Senapedis et al. (2023) ESMO World Congress on Gastrointestinal Cancer.

### Prophylactic Therapies

Also provided are one or more prophylactic treatments. In embodiments, a subject is administered a prophylactic treatment to reduce or eliminate an unintended effect of a therapy described herein. In embodiments, a prophylactic reduces or eliminates a side effect of gedatolisib, or pharmaceutically acceptable salt, solvate, or ester thereof. In embodiments, a prophylactic reduces or eliminates a side effect of darolutamide.

A prophylactic treatment of the disclosure can be administered prior, concurrent, or following administration of a therapy described herein.

### V. Treatment Regimens

The disclosure provides various treatment regimens that combine administration of gedatolisib, or a pharmaceutically acceptable salt, solvate, or ester thereof, with hormone therapy in a hormonally driven disorder other than breast cancer, optionally with one or more additional therapies added to the combination, as described herein.

In embodiments, gedatolisib, or a pharmaceutically acceptable salt, solvate, or ester thereof, is administered on a weekly basis, for two or more weeks, and the hormone therapy is administered according to its recommended treatment schedule. For example, for hormone therapy that uses an ARsi having a recommended treatment schedule of daily administration, the hormone therapy is administered daily in combination with the weekly administration of gedatolisib, or a pharmaceutically acceptable salt, solvate, or ester thereof. Similarly, for hormone therapy using an ARsi having a recommended treatment schedule of twice daily administration (e.g., darolutamide), the hormone therapy is administered twice daily in combination with the weekly administration of gedatolisib, or a pharmaceutically acceptable salt, solvate, or ester thereof. And the like for other hormone therapies with other recommended treatment schedules, combined with weekly gedatolisib treatment. In embodiments, one or more additional therapies are added to the combination as described in Subsection IV, wherein the one or more additional therapies are also used according to their recommended treatment schedule(s).

In other embodiments, gedatolisib, or a pharmaceutically acceptable salt, solvate, or ester thereof, is administered according to a three week on/one week off treatment schedule that is carried out for two or more 28-day (4 week) cycles. Thus, for each 28-day cycle, gedatolisib, or a pharmaceutically acceptable salt, solvate, or ester thereof, is administered on Day 1, Day 8 and Day 15 of the cycle, but not on Day 22 of the cycle, and then weekly administration begins again on Day 29 (Day 1 of the next cycle).

Accordingly, in embodiments, the gedatolisib treatment regimen used in a combination method of the disclosure comprises:
administering to the subject a therapeutically effective amount of gedatolisib, or a pharmaceutically acceptable salt, solvate, or ester thereof, once a week for an administration period of three weeks;
discontinuing administration of gedatolisib, or pharmaceutically acceptable salt, solvate, or ester thereof, for a discontinuation period of one week; and
resuming administration of gedatolisib, or pharmaceutically acceptable salt, solvate, or ester thereof, once a week following the discontinuation period,
wherein the administration period of three weeks and the discontinuation period of one week constitute a cycle, wherein the cycle is repeated for at least two cycles.

When a 3 week on/1 week off schedule is used for gedatolisib treatment, in one embodiment, the hormone therapy is administered according to its recommended treatment schedule (e.g., daily ARsi administration for an ARsi recommended for daily use; twice daily ARsi administration for an ARsi recommended for twice daily use, such as darolutamide).

When a 3 week on/1 week off schedule is used for gedatolisib treatment, in another embodiment, the hormone therapy treatment schedule is also discontinued for the 1 week off period to thereby synchronize (i.e., coordinate) the gedatolisib treatment schedule and the hormone therapy treatment schedule. The hormone therapy is used according to its recommended treatment schedule for the 3 weeks on period and then discontinued for the 1 week off period. Thus, for example, for an ARsi recommended for daily use, the ARsi is administered daily on Days 1-21 of the 28-day cycle and then discontinued for Days 22-28 of the 28 day cycle such that the period of discontinuation is concurrent for the gedatolisib treatment and the hormone therapy. Similarly, for an ARsi recommended for twice daily use (e.g., darolutamide), the ARsi is administered twice daily on Days 1-21 of the 28-day cycle and then discontinued for Days 22-28 of the cycle. And the like for other hormone therapies with other recommended treatment schedules, with the 1 week discontinuation period of the hormone therapy being concurrent with the 1 week discontinuation period of gedatolisib treatment.

When a 3 week on/1 week off schedule is used for gedatolisib treatment and one or more additional therapies as described in Subsection IV are combined in the method, in one embodiment the additional therapy is administered according to its recommended treatment schedule.

When a 3 week on/1 week off schedule is used for gedatolisib treatment and one or more additional therapies as described in Subsection IV are combined in the method, in another embodiment the additional therapy treatment schedule is also discontinued for the 1 week off discontinuation period to thereby synchronize (i.e., coordinate) the gedatolisib treatment schedule and the additional therapy treatment schedule. The additional therapy is used according to its recommended treatment schedule for the 3 weeks on period and then discontinued for the 1 week off period. Thus, for example, for an additional therapy recommended for daily use, the additional therapy is administered daily on Days 1-21 of the 28-day cycle and then discontinued for Days 22-28 of the 28-day cycle such that the period of discontinuation is concurrent for the gedatolisib treatment and the additional therapy. For an additional therapy with a recommended treatment schedule of 3 weeks on/1 week off, such as the CDK 4/6 inhibitor palbociclib, the additional therapy is administered on Days 1, 8 and 15 but not on Day 22 of the 28 day cycle, to thereby synchronize the gedatolisib treatment schedule and the additional therapy treatment schedule, with the 1 week discontinuation period of the additional therapy being concurrent with the 1 week discontinuation period of gedatolisib treatment.

Accordingly, in embodiments, the disclosure pertains to a method of treating a hormonally driven disorder in a human subject, the method comprising administering to a human subject with a hormonally driven disorder:
gedatolisib, or a pharmaceutically acceptable salt, solvate, or ester thereof, at a dosage of 100-300 mg weekly for at least two weeks; and
a hormone therapy for at least two weeks;
wherein the hormonally driven disorder is not breast cancer.

In embodiments, the dosage of gedatolisib, or a pharmaceutically acceptable salt, solvate, or ester thereof, is 100 mg, 120 mg, 150 mg, 180 mg, 210 mg, 240 mg, 270 or 300 mg weekly. In embodiments, administration of gedatolisib, or a pharmaceutically acceptable salt, solvate, or ester thereof, and hormone therapy is performed for three weeks, four weeks, five weeks, six weeks, seven weeks, eight weeks or longer, as discussed in Subsections II and III above. In embodiments, the hormone therapy comprises a therapy set forth in Subsection III above, including recommended dosages. In embodiments, the hormonally driven disorder comprises a disorder set forth in Subsection I above.

In another aspect, the disclosure provides treatment regimens for the treatment of prostate cancer. Accordingly, in embodiments, the disclosure pertains to a method of treating prostate cancer in a human subject, the method comprising administering to a human subject with prostate cancer:
gedatolisib, or a pharmaceutically acceptable salt, solvate, or ester thereof, at a dosage of 100-300 mg weekly for at least two weeks; and
a hormone therapy for at least two weeks.

In embodiments, the dosage of gedatolisib, or a pharmaceutically acceptable salt, solvate, or ester thereof, is 100 mg, 120 mg, 150 mg, 180 mg, 210 mg, 240 mg, 270 or 300 mg weekly. In embodiments, administration of gedatolisib, or a pharmaceutically acceptable salt, solvate, or ester thereof, and hormone therapy is performed for three weeks, four weeks, five weeks, six weeks, seven weeks, eight weeks or longer, as discussed in Subsections II and III above. In embodiments, the hormone therapy comprises a therapy set forth in Subsection III above, including recommended dosages. In embodiments, the prostate cancer is selected from the group consisting of adenocarcinoma of the prostate, transitional cell carcinoma of the prostate, squamous cell carcinoma of the prostate and small cell prostate cancer.

In embodiments for prostate cancer treatment, the hormone therapy comprises administration of darolutamide according to its recommended dosage and treatment schedule. Thus, in embodiments, the disclosure pertains to a method of treating prostate cancer in a human subject, the method comprising administering to a human subject with prostate cancer:
gedatolisib, or a pharmaceutically acceptable salt, solvate, or ester thereof, at a dosage of 100-300 mg weekly; and
darolutamide at a dosage of 600 mg twice daily.

In embodiments, the dosage of gedatolisib, or a pharmaceutically acceptable salt, solvate, or ester thereof, is 100 mg, 120 mg, 150 mg, 180 mg, 210 mg or 240 mg weekly. In embodiments, administration of gedatolisib, or a pharmaceutically acceptable salt, solvate, or ester thereof, and darolutamide is performed for three weeks, four weeks, five weeks, six weeks, seven weeks, eight weeks or longer, as discussed in Subsections II and III above. In embodiments, the prostate cancer is selected from the group consisting of adenocarcinoma of the prostate, transitional cell carcinoma of the prostate, squamous cell carcinoma of the prostate and small cell prostate cancer.

In another aspect, the disclosure provides treatment regimens for the treatment of endometrial disorders. Accordingly, in embodiments, the disclosure pertains to a method of treating an endometrial disorder in a human subject, the method comprising administering to a human subject with an endometrial disorder:
gedatolisib, or a pharmaceutically acceptable salt, solvate, or ester thereof, at a dosage of 100-300 mg weekly for at least two weeks; and
a hormone therapy for at least two weeks;
wherein the endometrial disorder is selected from the group consisting of endometriosis, endometrial hyperplasia, endometrial carcinoma, endometrial intraepithelial neoplasia (EIN), intrauterine adhesions (IUA), adenomyosis, endometritis, endometrial and/or uterine polyps and Asherman syndrome.

In embodiments, the dosage of gedatolisib, or a pharmaceutically acceptable salt, solvate, or ester thereof, is 100 mg, 120 mg, 150 mg, 180 mg, 210 mg, 240 mg, 270 or 300 mg weekly. In embodiments, administration of gedatolisib, or a pharmaceutically acceptable salt, solvate, or ester thereof, and hormone therapy is performed for three weeks, four weeks, five weeks, six weeks, seven weeks, eight weeks or longer, as discussed in Subsections II and III above. In embodiments, the hormone therapy comprises a therapy set forth in Subsection III above, including recommended dosages. In embodiments, the hormone therapy comprises administration of letrozole according to its recommended dosage and treatment schedule. In embodiments, the hormone therapy comprises administration of fulvestrant according to its recommended dosage and treatment schedule. In embodiments, the hormone therapy comprises administration of tamoxifen according to its recommended dosage and treatment schedule. In embodiments, treatment of the endometrial disorder further includes administration to the subject of a CDK 4/6 inhibitor, as discussed in Subsection IV above. In embodiments, the CDK 4/6 inhibitor is palbociclib, which is used according to its recommended dosage and treatment schedule.

Regarding timing and length of treatment, the method comprising administering gedatolisib, or a pharmaceutically acceptable salt, solvate, or ester thereof, and a hormone therapy (and optionally one or more additional treatments) are carried out for at least two weeks and typically are carried out for longer, such as multiple four-week (28 day) cycles.

In certain embodiments, gedatolisib, or a pharmaceutically acceptable salt, solvate, or ester thereof, is administered on a weekly basis (i.e., once a week) without interruption during the length of the treatment period.

In other embodiments, as described in detail above, gedatolisib, or a pharmaceutically acceptable salt, solvate, or ester thereof, is administered weekly (i.e., once a week) on a three week on/one week off schedule during the length of the treatment period. Thus, for a 28-day treatment cycle, gedatolisib, or a pharmaceutically acceptable salt, solvate, or ester thereof, is administered on Days 1, 8 and 15 but not on Day 22 and then resumes again on Day 29 (i.e., Day 1 of the next 28-day cycle).

Accordingly, in embodiments, a method of the disclosure combines hormone therapy with administration of gedatolisib, or pharmaceutically acceptable salt, solvate, or ester thereof, in a method of treating a hormonally driven disorder other than breast cancer comprising:
administering to a human subject with a hormonally driven disorder other than breast cancer:
gedatolisib, or a pharmaceutically acceptable salt, solvate, or ester thereof, at a dosage of 100-300 mg once a week for an administration period of three weeks;
discontinuing administration of gedatolisib, or pharmaceutically acceptable salt, solvate, or ester thereof, for a discontinuation period of one week; and
resuming administration of gedatolisib, or pharmaceutically acceptable salt, solvate, or ester thereof, once a week following the discontinuation period,
wherein the administration period of three weeks and the discontinuation period of one week constitutes a cycle, wherein the cycle is repeated for at least two cycles; and
a hormone therapy that is administered for the at least two cycles.

In embodiments, the hormone therapy is administered according to its recommended treatment schedule (e.g., twice daily for darolutamide) for the entire treatment period.

In embodiments, the hormone therapy is administered according to its recommended treatment schedule for an administration period of three weeks and then administration is discontinued for a discontinuation period of one week, wherein the discontinuation period is concurrent with (synchronized with) the discontinuation period of gedatolisib treatment.

In embodiments, the method further comprises administering one or more additional therapies (e.g., as in Subsection IV) to the subject. In embodiments, the additional therapy is administered according to its recommended treatment schedule for the entire treatment period. In embodiments, the additional therapy is administered according to its recommended treatment schedule for an administration period of three weeks and then administration is discontinued for a discontinuation period of one week, wherein the discontinuation period is concurrent with (synchronized with) the discontinuation period of gedatolisib treatment.

In another embodiment, the disclosure pertains to a method of treating prostate cancer comprising administering to a human subject with prostate cancer:
(a) gedatolisib, or a pharmaceutically acceptable salt, solvate, or ester thereof, at a dosage of 100-300 mg once a week for an administration period of three weeks;
discontinuing administration of gedatolisib, or pharmaceutically acceptable salt, solvate, or ester thereof, for a discontinuation period of one week; and
resuming administration of gedatolisib, or pharmaceutically acceptable salt, solvate, or ester thereof, once a week following the discontinuation period; and
(b) darolutamide at a dosage of 600 mg twice daily for an administration period of three weeks;
discontinuing administration of darolutamide for a discontinuation period of one week; and
resuming administration of darolutamide at a dosage of 600 mg twice daily following the discontinuation period;
wherein the administration period of three weeks and the discontinuation period of one week constitutes a cycle, wherein the cycle is repeated for at least two cycles; and
wherein the discontinuation period for administering gedatolisib, or a pharmaceutically acceptable salt, solvate, or ester thereof, and the discontinuation period for administering darolutamide are concurrent.

In embodiments, the dosage of gedatolisib, or a pharmaceutically acceptable salt, solvate, or ester thereof, is 100 mg, 120 mg, 150 mg, 180 mg, 210 mg, 240 mg, 270 or 300 mg weekly. In embodiments, three, four, five, six, seven, eight, nine, ten or more cycles of treatment are performed.

In certain embodiments in which gedatolisib, or a pharmaceutically acceptable salt, solvate, or ester thereof, is administered on a three week one/one week off schedule, its administration is synchronized with (coordinated with) another therapy, such as treatment with a CDK 4/6 inhibitor that is also administered according to a three week on/one week off treatment schedule, such as palbociclib (as discussed above). Accordingly, in an embodiment, gedatolisib, or a pharmaceutically acceptable salt, solvate, or ester thereof, is administered on a three week on/one week off schedule (Days 1, 8 and 15 but not 22) and the subject is also treated with a CDK 4/6 inhibitor such as palbociclib, which for a 28 day cycle is also administered according to a three week on/one week off schedule (Days 1, 8 and 15 but not 22).

Accordingly, in another embodiment, the disclosure pertains to a method of treating an endometrial disorder comprising administering to a human subject with an endometrial disorder:
(a) gedatolisib, or a pharmaceutically acceptable salt, solvate, or ester thereof, at a dosage of 100-300 mg once a week for an administration period of three weeks;
discontinuing administration of gedatolisib, or pharmaceutically acceptable salt, solvate, or ester thereof, for a discontinuation period of one week; and
resuming administration of gedatolisib, or pharmaceutically acceptable salt, solvate, or ester thereof, once a week following the discontinuation period;
(b) a hormone therapy; and
(c) a CDK4/6 inhibitor for an administration period of three weeks;
discontinuing administration of the CDK4/6 inhibitor for a discontinuation period of one week; and
resuming administration of the CDK4/6 inhibitor following the discontinuation period;
wherein the administration period of three weeks and the discontinuation period of one week constitutes a cycle, wherein the cycle is repeated for at least two cycles;
wherein the discontinuation period for administering gedatolisib, or a pharmaceutically acceptable salt, solvate, or ester thereof, and the discontinuation period for administering the CDK 4/6 inhibitor are concurrent; and
wherein the endometrial disorder is selected from the group consisting of endometriosis, endometrial hyperplasia, endometrial carcinoma, endometrial intraepithelial neoplasia (EIN), intrauterine adhesions (IUA), adenomyosis, endometritis, endometrial and/or uterine polyps and Asherman syndrome.

In embodiments, the dosage of gedatolisib, or a pharmaceutically acceptable salt, solvate, or ester thereof, is 100 mg, 120 mg, 150 mg, 180 mg, 210 mg or 240 mg weekly. Non-limiting examples of suitable hormone therapies are set forth in Subsection III above. In embodiments, the hormone therapy is an anti-estrogen therapy, such as administration of letrozole or tamoxifen as described herein. Non-limiting examples of suitable CDK4/6 inhibitors are set forth in Subsection IV above. In embodiments, the CDK4/6 inhibitor is palbociclib, as described hereon.

In embodiments, three, four, five, six, seven, eight, nine, ten or more cycles of treatment are performed.

### VI. Kits

Provided are also kits comprising Gedatolisib, or a pharmaceutically acceptable salt, solvate, or ester thereof; and a hormone therapy. In embodiments, a kit comprises Gedatolisib, or a pharmaceutically acceptable salt, solvate, or ester thereof; and a hormone therapy selected from the group consisting of: darolutamide, apalutamide, bicalutamide, enzalutamide, flutamide, nilutamide and topilutamide. In embodiments, provided is a kit comprising Gedatolisib, or a pharmaceutically acceptable salt, solvate, or ester thereof; and an ARsi. In embodiments the ARsi is darolutamide.

Provided are also kits comprising Gedatolisib, or a pharmaceutically acceptable salt, solvate, or ester thereof; and a SERM selected from the group consisting of: tamoxifen, raloxifene, toremifene, ospemifene, bazedoxifene, ormeloxifene, lasofoxifene, cyclofenil, clomifene, broparestrol and anordin.

Provided are also kits comprising Gedatolisib, or a pharmaceutically acceptable salt, solvate, or ester thereof; and a SERD selected from the group consisting of: fulvestrant, elacestrant, amcenestrant, camizestrant, giredestrant, imlunestrant, vepdegestrant, palazestrant, LSZ102, rintodestrant, SHR9549, H3B-5942, ZN-c5, and brilanestrant.

Provided are also kits comprising Gedatolisib, or a pharmaceutically acceptable salt, solvate, or ester thereof; and an aromastase inhibitor selected from the group consisting of letrozole, anastrozole and exemestane.

Provided are also kits comprising Gedatolisib, or a pharmaceutically acceptable salt, solvate, or ester thereof; a hormone therapy; and a CDK4/6 inhibitor.

Kits of the disclosure can comprise instructions for administration of any of the therapeutics of the disclosure.

### Additional Embodiments

In other embodiments, the disclosure provides gedatolisib, or a pharmaceutically acceptable salt, solvate, or ester thereof, for use in a method of the disclosure. Non-limiting examples of additional embodiments include the following:

1. Gedatolisib, or a pharmaceutically acceptable salt, solvate, or ester thereof, for use in a method of treating a hormonally driven disorder in a human subject, the method comprising:
administering the gedatolisib, or the pharmaceutically acceptable salt, solvate, or ester thereof, at a dosage of 100-300 mg weekly for at least two weeks; and
a hormone therapy for at least two weeks;
wherein the hormonally driven disorder is not breast cancer.
   2. Gedatolisib, or a pharmaceutically acceptable salt, solvate, or ester thereof, for use according to embodiment 1, wherein the hormonally driven disorder is prostate cancer.
   3. Gedatolisib, or a pharmaceutically acceptable salt, solvate, or ester thereof, for use according to embodiment 2, wherein the prostate cancer has become hormone resistant at time of administering gedatolisib, or a pharmaceutically acceptable salt, solvate, or ester thereof.
   4. Gedatolisib, or a pharmaceutically acceptable salt, solvate, or ester thereof, for use according to embodiment 2, wherein the hormone therapy comprises administering to the subject an androgen receptor signaling inhibitor (ARsi).
   5. Gedatolisib, or a pharmaceutically acceptable salt, solvate, or ester thereof, for use according to embodiment 4, wherein the ARsi comprises a non-steroidal androgen receptor inhibitor selected from the group consisting of darolutamide, apalutamide, bicalutamide, enzalutamide, flutamide, nilutamide and topilutamide.
   6. Gedatolisib, or a pharmaceutically acceptable salt, solvate, or ester thereof, for use according to embodiment 5, wherein the ARsi is darolutamide.
   7. Gedatolisib, or a pharmaceutically acceptable salt, solvate, or ester thereof, for use according to embodiment 6, wherein darolutamide is orally administered at a dosage of 600 mg twice daily.
   8. Gedatolisib, or a pharmaceutically acceptable salt, solvate, or ester thereof, for use according to embodiment 2, wherein the hormone therapy comprises administering to the subject a steroidal anti-androgen, an androgen synthesis inhibitor, an androgen receptor pathway inhibitor, an androgen receptor protein degrader, or an N-terminal domain (NTD) androgen receptor inhibitor.
   9. Gedatolisib, or a pharmaceutically acceptable salt, solvate, or ester thereof, for use according to embodiment 8, wherein the steroidal anti-androgen is a progesterone derivative, a testosterone derivative, a spirolactone derivative or a cortisol derivative.
   10. Gedatolisib, or a pharmaceutically acceptable salt, solvate, or ester thereof, for use according to embodiment 9, wherein the progesterone derivative is selected from the group consisting of 11α-hydroxyprogesterone, Chlormadinone acetate, Clometerone, Cyproterone. Cyproterone acetate, Edogestrone, Medrogestone, Megestrol acetate, Nomegestrol acetate and Osaterone acetate.
   11. Gedatolisib, or a pharmaceutically acceptable salt, solvate, or ester thereof, for use according to embodiment 9, wherein the testosterone derivative is selected from the group consisting of Abiraterone, Abiraterone acetate, Benorterone, BOMT, Delanterone, Dienogest, Epitestosterone, Galeterone, Metogest, Oxendolone, Rosterolone, Topterone, Trimethyltrienolone and Zanoterone.
   12. Gedatolisib, or a pharmaceutically acceptable salt, solvate, or ester thereof, for use according to embodiment 9, wherein the spirolactone derivative is selected from the group consisting of SC-5233, SC-8109, Canrenone, Dicirenone, Drospirenone, Mespirenone, Mexrenone, Prorenone, Spironolactone, Spirorenone and Spiroxasone.
   13. Gedatolisib, or a pharmaceutically acceptable salt, solvate, or ester thereof, for use according to embodiment 9, wherein the cortisol derivative is selected from the group consisting of 9,11-Dehydrocortexolone 17α-butyrate and Clascoterone.
   14. Gedatolisib, or a pharmaceutically acceptable salt, solvate, or ester thereof, for use according to embodiment 8, wherein the androgen synthesis inhibitor is selected from the group consisting of finasteride, dutasteride and leuprolide.
   15. Gedatolisib, or a pharmaceutically acceptable salt, solvate, or ester thereof, for use according to embodiment 8, wherein the androgen receptor pathway inhibitor is abiraterone acetate.
   16. Gedatolisib, or a pharmaceutically acceptable salt, solvate, or ester thereof, for use according to embodiment 1, wherein the hormonally driven disorder is hormonally driven disorder is endometrial cancer, uterine cancer or ovarian cancer.
   17. Gedatolisib, or a pharmaceutically acceptable salt, solvate, or ester thereof, for use according to embodiment 1, wherein the hormonally driven disorder is an endometrial disorder.
   18. Gedatolisib, or a pharmaceutically acceptable salt, solvate, or ester thereof, for use according to embodiment 17, wherein the endometrial disorder is selected from the group consisting of endometriosis, endometrial hyperplasia, endometrial carcinoma, endometrial intraepithelial neoplasia (EIN), intrauterine adhesions (IUA), adenomyosis, endometritis, endometrial and/or uterine polyps and Asherman syndrome.
   19. Gedatolisib, or a pharmaceutically acceptable salt, solvate, or ester thereof, for use according to embodiment 18, wherein the endometrial disorder is endometriosis.
   20. Gedatolisib, or a pharmaceutically acceptable salt, solvate, or ester thereof, for use according to any one of embodiments 1-19, wherein gedatolisib, or a pharmaceutically acceptable salt, solvate, or ester thereof, is administered at a dosage of 100 mg weekly.
   21. Gedatolisib, or a pharmaceutically acceptable salt, solvate, or ester thereof, for use according to any one of embodiments 1-19, wherein gedatolisib, or a pharmaceutically acceptable salt, solvate, or ester thereof, is administered at a dosage of 120 mg weekly.
   22. Gedatolisib, or a pharmaceutically acceptable salt, solvate, or ester thereof, for use according to any one of embodiments 1-19, wherein gedatolisib, or a pharmaceutically acceptable salt, solvate, or ester thereof, is administered at a dosage of 150 mg weekly.
   23. Gedatolisib, or a pharmaceutically acceptable salt, solvate, or ester thereof, for use according to according to any one of embodiments 1-19, wherein gedatolisib, or a pharmaceutically acceptable salt, solvate, or ester thereof, is administered at a dosage of 180 mg weekly.
   24. Gedatolisib, or a pharmaceutically acceptable salt, solvate, or ester thereof, for use according to any one of embodiments 1-19, wherein gedatolisib, or a pharmaceutically acceptable salt, solvate, or ester thereof, is administered at a dosage of 210 mg weekly.
   25. Gedatolisib, or a pharmaceutically acceptable salt, solvate, or ester thereof, for use according to any one of embodiments 1-19, wherein gedatolisib, or a pharmaceutically acceptable salt, solvate, or ester thereof, is administered at a dosage of 240 mg weekly.
   26. Gedatolisib, or a pharmaceutically acceptable salt, solvate, or ester thereof, for use according to any one of embodiments 1-25, wherein gedatolisib, or a pharmaceutically acceptable salt, solvate, or ester thereof, and the hormone therapy are administered for at least four weeks.
   27. Gedatolisib, or a pharmaceutically acceptable salt, solvate, or ester thereof, for use according to any one of embodiments 1-26, which further comprises treating the human subject with one or more additional therapies effective in the hormonally driven disorder.
   28. Gedatolisib, or a pharmaceutically acceptable salt, solvate, or ester thereof, for use according to any one of embodiments 27, wherein the one or more additional therapies comprise administering to the subject one or more chemotherapeutic drugs or therapies that target oncogenic proteins.
   29. Gedatolisib, or a pharmaceutically acceptable salt, solvate, or ester thereof, for use according to embodiment 27, wherein the one or more additional therapies comprise administering to the subject one or more immune checkpoint inhibitors.
   30. Gedatolisib, or a pharmaceutically acceptable salt, solvate, or ester thereof, for use according to embodiment 27, wherein the one or more additional therapies comprise one or more surgical treatments.
   31. Gedatolisib, or a pharmaceutically acceptable salt, solvate, or ester thereof, for use according to embodiment 27, wherein the one or more additional therapies comprise administering to the subject one or more receptor tyrosine kinase (RTK) inhibitors.
   32. Gedatolisib, or a pharmaceutically acceptable salt, solvate, or ester thereof, for use according to embodiment 27, wherein the one or more additional therapies comprise administering to the subject one or more CDK 4/6 inhibitors.
   33. Gedatolisib, or a pharmaceutically acceptable salt, solvate, or ester thereof, for use according to embodiment 27, wherein the one or more additional therapies comprise administering to the subject one or more epigenetic-targeted therapies.
   34. Gedatolisib, or a pharmaceutically acceptable salt, solvate, or ester thereof, for use according to embodiment 33, wherein the epigenetic targeted therapy targets an epigenetic regulator selected from the group consisting of histone deacetylase (HDAC), lysine acetyltransferase 6 (KAT6), DNA methyltransferase (DNMT), histone methyltransferase (HMT), Bromodomain and Extra-Terminal motif (BET) and histone lysine demethylase.
   35. Gedatolisib, or a pharmaceutically acceptable salt, solvate, or ester thereof, for use in a method of treating prostate cancer in a human subject, the method comprising:
administering to a human subject with prostate cancer:
   gedatolisib, or a pharmaceutically acceptable salt, solvate, or ester thereof, at a dosage of 100-300 mg weekly for at least two weeks; and
      a hormone therapy for at least two weeks.
         36. Gedatolisib, or a pharmaceutically acceptable salt, solvate, or ester thereof, for use according to embodiment 35, wherein the prostate cancer is hormone driven at time of administering gedatolisib, or a pharmaceutically acceptable salt, solvate, or ester thereof.
         37. Gedatolisib, or a pharmaceutically acceptable salt, solvate, or ester thereof, for use according to embodiment 35, wherein the prostate cancer has become hormone resistant at time of administering gedatolisib, or a pharmaceutically acceptable salt, solvate, or ester thereof.
         38. Gedatolisib, or a pharmaceutically acceptable salt, solvate, or ester thereof, for use according to embodiment 35, wherein the hormone therapy comprises administering to the subject an androgen receptor signaling inhibitor (ARsi).
         39. Gedatolisib, or a pharmaceutically acceptable salt, solvate, or ester thereof, for use according to embodiment 38, wherein the ARsi is a non-steroidal androgen receptor inhibitor is selected from the group consisting of darolutamide, apalutamide, bicalutamide, enzalutamide, flutamide, nilutamide and topilutamide.
         40. Gedatolisib, or a pharmaceutically acceptable salt, solvate, or ester thereof, for use according to embodiment 39, wherein the ARsi is darolutamide.
         41. Gedatolisib, or a pharmaceutically acceptable salt, solvate, or ester thereof, for use according to embodiment 40, wherein darolutamide is orally administered at a dosage of 600 mg twice daily.
         42. Gedatolisib, or a pharmaceutically acceptable salt, solvate, or ester thereof, for use according to embodiment 35, wherein the hormone therapy comprises administering to the subject a steroidal anti-androgen, an androgen synthesis inhibitor, an androgen receptor pathway inhibitor, an androgen receptor protein degrader, or an N-terminal domain (NTD) androgen receptor inhibitor.
         43. Gedatolisib, or a pharmaceutically acceptable salt, solvate, or ester thereof, for use according to embodiment 42, wherein the steroidal anti-androgen is a progesterone derivative, a testosterone derivative, a spirolactone derivative or a cortisol derivative.
         44. Gedatolisib, or a pharmaceutically acceptable salt, solvate, or ester thereof, for use according to embodiment 43, wherein the progesterone derivative is selected from the group consisting of 11α-hydroxyprogesterone, Chlormadinone acetate, Clometerone, Cyproterone, Cyproterone acetate, Edogestrone, Medrogestone, Megestrol acetate, Nomegestrol acetate and Osaterone acetate.
         45. Gedatolisib, or a pharmaceutically acceptable salt, solvate, or ester thereof, for use according to embodiment 43, wherein the testosterone derivative is selected from the group consisting of Abiraterone, Abiraterone acetate, Benorterone, BOMT, Delanterone, Dienogest. Epitestosterone, Galcterone, Metogest, Oxendolone, Rosterolone, Topterone, Trimethyltrienolone and Zanoterone.
         46. Gedatolisib, or a pharmaceutically acceptable salt, solvate, or ester thereof, for use according to embodiment 43, wherein the spirolactone derivative is selected from the group consisting of SC-5233, SC-8109, Canrenone, Dicirenone, Drospirenone, Mespirenone, Mexrenone, Prorenone, Spironolactone, Spirorenone and Spiroxasone.
         47. Gedatolisib, or a pharmaceutically acceptable salt, solvate, or ester thereof, for use according to embodiment 43, wherein the cortisol derivative is selected from the group consisting of 9,11-Dehydrocortexolone 17α-butyrate and Clascoterone.
         48. Gedatolisib, or a pharmaceutically acceptable salt, solvate, or ester thereof, for use according to embodiment 42, wherein the androgen synthesis inhibitor is selected from the group consisting of finasteride, dutasteride and leuprolide.
         49. Gedatolisib, or a pharmaceutically acceptable salt, solvate, or ester thereof, for use according to embodiment 42, wherein the androgen receptor pathway inhibitor is abiraterone acetate.
         50. Gedatolisib, or a pharmaceutically acceptable salt, solvate, or ester thereof, for use according to any one of embodiments 35-49, wherein gedatolisib, or a pharmaceutically acceptable salt, solvate, or ester thereof, is administered at a dosage of 100 mg weekly.
         51. Gedatolisib, or a pharmaceutically acceptable salt, solvate, or ester thereof, for use according to any one of embodiments 35-49, wherein gedatolisib, or a pharmaceutically acceptable salt, solvate, or ester thereof, is administered at a dosage of 120 mg weekly.
         52. Gedatolisib, or a pharmaceutically acceptable salt, solvate, or ester thereof, for use according to any one of embodiments 35-49, wherein gedatolisib, or a pharmaceutically acceptable salt, solvate, or ester thereof, is administered at a dosage of 150 mg weekly.
         53. Gedatolisib, or a pharmaceutically acceptable salt, solvate, or ester thereof, for use according to any one of embodiments 35-49, wherein gedatolisib, or a pharmaceutically acceptable salt, solvate, or ester thereof, is administered at a dosage of 180 mg weekly.
         54. Gedatolisib, or a pharmaceutically acceptable salt, solvate, or ester thereof, for use according to any one of embodiments 35-49, wherein gedatolisib, or a pharmaceutically acceptable salt, solvate, or ester thereof, is administered at a dosage of 210 mg weekly.
         55. Gedatolisib, or a pharmaceutically acceptable salt, solvate, or ester thereof, for use according to any one of embodiments 35-49, wherein gedatolisib, or a pharmaceutically acceptable salt, solvate, or ester thereof, is administered at a dosage of 240 mg weekly.
         56. Gedatolisib, or a pharmaceutically acceptable salt, solvate, or ester thereof, for use according to any one of embodiments 35-55, wherein gedatolisib, or a pharmaceutically acceptable salt, solvate, or ester thereof, and the hormone therapy are administered for at least four weeks.
         57. Gedatolisib, or a pharmaceutically acceptable salt, solvate, or ester thereof, for use according to any one of embodiments 35-56, which further comprises treating the human subject with one or more additional therapies effective in prostate cancer.
         58. Gedatolisib, or a pharmaceutically acceptable salt, solvate, or ester thereof, for use according to embodiment 57, wherein the one or more additional therapies comprise administering to the subject one or more chemotherapeutic drugs or therapies that target oncogenic proteins.
         59. Gedatolisib, or a pharmaceutically acceptable salt, solvate, or ester thereof, for use according to embodiment 57, wherein the one or more additional therapies comprise administering to the subject one or more immune checkpoint inhibitors.
         60. Gedatolisib, or a pharmaceutically acceptable salt, solvate, or ester thereof, for use according to embodiment 57, wherein the one or more additional therapies comprise one or more surgical treatments that lower testosterone.
         61. Gedatolisib, or a pharmaceutically acceptable salt, solvate, or ester thereof, for use according to embodiment 57, wherein the one or more additional therapies comprise administering to the subject one or more receptor tyrosine kinase (RTK) inhibitors.
         62. Gedatolisib, or a pharmaceutically acceptable salt, solvate, or ester thereof, for use according to embodiment 57, wherein the one or more additional therapies comprise administering to the subject one or more CDK 4/6 inhibitors.
         63. Gedatolisib, or a pharmaceutically acceptable salt, solvate, or ester thereof, for use according to embodiment 57, wherein the one or more additional therapies comprise administering to the subject one or more epigenetic-targeted therapies.
         64. Gedatolisib, or a pharmaceutically acceptable salt, solvate, or ester thereof, for use according to embodiment 63, wherein the epigenetic targeted therapy targets an epigenetic regulator selected from the group consisting of histone deacetylase (HDAC), lysine acetyltransferase 6 (KAT6), DNA methyltransferase (DNMT), histone methyltransferase (HMT), Bromodomain and Extra-Terminal motif (BET) and histone lysine demethylase.
         65. Gedatolisib, or a pharmaceutically acceptable salt, solvate, or ester thereof, for use in a method of treating prostate cancer in a human subject, the method comprising administering to a human subject with prostate cancer:
gedatolisib, or a pharmaceutically acceptable salt, solvate, or ester thereof, at a dosage of 100-300 mg weekly; and
   darolutamide at a dosage of 600 mg twice daily.
      66. Gedatolisib, or a pharmaceutically acceptable salt, solvate, or ester thereof, for use according to embodiment 65, wherein gedatolisib, or a pharmaceutically acceptable salt, solvate, or ester thereof, and darolutamide are administered to the subject for at least four weeks.
      67. Gedatolisib, or a pharmaceutically acceptable salt, solvate, or ester thereof, for use according to embodiment 65 or embodiment 66, which further comprises treating the human subject with one or more additional therapies effective in prostate cancer.
      68. Gedatolisib, or a pharmaceutically acceptable salt, solvate, or ester thereof, for use according to embodiment 67, wherein the one or more additional therapies comprise administering to the subject one or more chemotherapeutic drugs or therapies that target oncogenic proteins.
      69. Gedatolisib, or a pharmaceutically acceptable salt, solvate, or ester thereof, for use according to embodiment 67, wherein the one or more additional therapies comprise administering to the subject one or more immune checkpoint inhibitors.
      70. Gedatolisib, or a pharmaceutically acceptable salt, solvate, or ester thereof, for use according to embodiment 67, wherein the one or more additional therapies comprise one or more surgical treatments that lower testosterone.
      71. Gedatolisib, or a pharmaceutically acceptable salt, solvate, or ester thereof, for use according to embodiment 68, wherein the one or more additional therapies comprise administering to the subject one or more receptor tyrosine kinase (RTK) inhibitors.
      72. Gedatolisib, or a pharmaceutically acceptable salt, solvate, or ester thereof, for use according to embodiment 67, wherein the one or more additional therapies comprise administering to the subject one or more CDK 4/6 inhibitors.
      73. Gedatolisib, or a pharmaceutically acceptable salt, solvate, or ester thereof, for use according to embodiment 67, wherein the one or more additional therapies comprise administering to the subject one or more epigenetic-targeted therapies.
      74. Gedatolisib, or a pharmaceutically acceptable salt, solvate, or ester thereof, for use according to embodiment 73, wherein the epigenetic targeted therapy targets an epigenetic regulator selected from the group consisting of histone deacetylase (HDAC), lysine acetyltransferase 6 (KAT6), DNA methyltransferase (DNMT), histone methyltransferase (HMT), Bromodomain and Extra-Terminal motif (BET) and histone lysine demethylase.
      75. Gedatolisib, or a pharmaceutically acceptable salt, solvate, or ester thereof, for use according to any one of embodiments 67-74, wherein gedatolisib, or a pharmaceutically acceptable salt, solvate, or ester thereof, is administered at a dosage of 100 mg weekly.
      76. Gedatolisib, or a pharmaceutically acceptable salt, solvate, or ester thereof, for use according to any one of embodiments 67-74, wherein gedatolisib, or a pharmaceutically acceptable salt, solvate, or ester thereof, is administered at a dosage of 120 mg weekly.
      77. Gedatolisib, or a pharmaceutically acceptable salt, solvate, or ester thereof, for use according to any one of embodiments 67-74, wherein gedatolisib, or a pharmaceutically acceptable salt, solvate, or ester thereof, is administered at a dosage of 150 mg weekly.
      78. Gedatolisib, or a pharmaceutically acceptable salt, solvate, or ester thereof, for use according to any one of embodiments 67-74, wherein gedatolisib, or a pharmaceutically acceptable salt, solvate, or ester thereof, is administered at a dosage of 180 mg weekly.
      79. Gedatolisib, or a pharmaceutically acceptable salt, solvate, or ester thereof, for use according to any one of embodiments 67-74, wherein gedatolisib, or a pharmaceutically acceptable salt, solvate, or ester thereof, is administered at a dosage of 210 mg weekly.
      80. Gedatolisib, or a pharmaceutically acceptable salt, solvate, or ester thereof, for use according to any one of embodiments 67-74, wherein gedatolisib, or a pharmaceutically acceptable salt, solvate, or ester thereof, is administered at a dosage of 240 mg weekly.
      81. Gedatolisib, or a pharmaceutically acceptable salt, solvate, or ester thereof, for use in a method of treating an endometrial disorder in a human subject, the method comprising administering to a human subject with an endometrial disorder:
gedatolisib, or a pharmaceutically acceptable salt, solvate, or ester thereof, at a dosage of 100-300 mg weekly for at least two weeks; and
   a hormone therapy for at least two weeks;
wherein the endometrial disorder is selected from the group consisting of endometriosis, endometrial hyperplasia, endometrial carcinoma, endometrial intraepithelial neoplasia (EIN), intrauterine adhesions (IUA), adenomyosis, endometritis, endometrial and/or uterine polyps and Asherman syndrome.
   82. Gedatolisib, or a pharmaceutically acceptable salt, solvate, or ester thereof, for use according to embodiment 81, wherein the endometrial disorder is endometriosis.
   83. Gedatolisib, or a pharmaceutically acceptable salt, solvate, or ester thereof, for use according to embodiment 81, wherein the hormone therapy comprises administering to the subject a selective estrogen receptor modulator (SERM).
   84. Gedatolisib, or a pharmaceutically acceptable salt, solvate, or ester thereof, for use according to embodiment 83, wherein the SERM is selected from the group consisting of tamoxifen, raloxifene, toremifene, ospemifene, bazedoxifene, ormeloxifene, lasofoxifene, cyclofenil, clomifene, broparestrol and anordin.
   85. Gedatolisib, or a pharmaceutically acceptable salt, solvate, or ester thereof, for use according to embodiment 81, wherein the hormone therapy comprises administering to the subject a selective estrogen receptor degrader (SERD).
   86. Gedatolisib, or a pharmaceutically acceptable salt, solvate, or ester thereof, for use according to embodiment 85, wherein the SERD is selected from the group consisting of fulvestrant, elacestrant, amcenestrant, camizestrant, giredestrant, imlunestrant, vepdegestrant, palazestrant, LSZ102, rintodestrant, SHR9549, H3B-5942, ZN-c5, and brilanestrant.
   87. Gedatolisib, or a pharmaceutically acceptable salt, solvate, or ester thereof, for use according to embodiment 81, wherein the hormone therapy comprises administering to the subject an aromatase inhibitor.
   88. Gedatolisib, or a pharmaceutically acceptable salt, solvate, or ester thereof, for use according to embodiment 87, wherein the aromatase inhibitor is selected from the group consisting of letrozole, anastrozole and exemestane.
   89. Gedatolisib, or a pharmaceutically acceptable salt, solvate, or ester thereof, for use according to embodiment 81, wherein gedatolisib, or a pharmaceutically acceptable salt, solvate, or ester thereof, and the hormone therapy are administered to the subject for at least four weeks.
   90. Gedatolisib, or a pharmaceutically acceptable salt, solvate, or ester thereof, for use according to any one of embodiments 81-89, which further comprises treating the human subject with one or more additional therapies effective in the endometrial disorder.
   91. Gedatolisib, or a pharmaceutically acceptable salt, solvate, or ester thereof, for use according to embodiment 90, wherein the one or more additional therapies comprise administering to the subject one or more chemotherapeutic drugs or therapies that target oncogenic proteins.
   92. Gedatolisib, or a pharmaceutically acceptable salt, solvate, or ester thereof, for use according to embodiment 90, wherein the one or more additional therapies comprise administering to the subject one or more immune checkpoint inhibitors.
   93. Gedatolisib, or a pharmaceutically acceptable salt, solvate, or ester thereof, for use according to embodiment 90, wherein the one or more additional therapies comprise one or more surgical treatments that alters hormones in the subject.
   94. Gedatolisib, or a pharmaceutically acceptable salt, solvate, or ester thereof, for use according to embodiment 90, wherein the one or more additional therapies comprise administering to the subject one or more receptor tyrosine kinase (RTK) inhibitors.
   95. Gedatolisib, or a pharmaceutically acceptable salt, solvate, or ester thereof, for use according to embodiment 90, wherein the one or more additional therapies comprise administering to the subject one or more CDK 4/6 inhibitors.
   96. Gedatolisib, or a pharmaceutically acceptable salt, solvate, or ester thereof, for use according to embodiment 90, wherein the one or more additional therapies comprise administering to the subject one or more epigenetic-targeted therapies.
   97. Gedatolisib, or a pharmaceutically acceptable salt, solvate, or ester thereof, for use according to embodiment 96, wherein the epigenetic targeted therapy targets an epigenetic regulator selected from the group consisting of histone deacetylase (HDAC), lysine acetyltransferase 6 (KAT6), DNA methyltransferase (DNMT), histone methyltransferase (HMT), Bromodomain and Extra-Terminal motif (BET) and histone lysine demethylase.
   98. Gedatolisib, or a pharmaceutically acceptable salt, solvate, or ester thereof, for use according to any one of embodiments 81-97, wherein gedatolisib, or a pharmaceutically acceptable salt, solvate, or ester thereof, is administered at a dosage of 100 mg weekly.
   99. Gedatolisib, or a pharmaceutically acceptable salt, solvate, or ester thereof, for use according to any one of embodiments 81-97, wherein gedatolisib, or a pharmaceutically acceptable salt, solvate, or ester thereof, is administered at a dosage of 120 mg weekly.
   100. Gedatolisib, or a pharmaceutically acceptable salt, solvate, or ester thereof, for use according to any one of embodiments 81-97, wherein gedatolisib, or a pharmaceutically acceptable salt, solvate, or ester thereof, is administered at a dosage of 150 mg weekly.
   101. Gedatolisib, or a pharmaceutically acceptable salt, solvate, or ester thereof, for use according to any one of embodiments 81-97, wherein gedatolisib, or a pharmaceutically acceptable salt, solvate, or ester thereof, is administered at a dosage of 180 mg weekly.
   102. Gedatolisib, or a pharmaceutically acceptable salt, solvate, or ester thereof, for use according to any one of embodiments 81-97, wherein gedatolisib, or a pharmaceutically acceptable salt, solvate, or ester thereof, is administered at a dosage of 210 mg weekly.
   103. Gedatolisib, or a pharmaceutically acceptable salt, solvate, or ester thereof, for use according to any one of embodiments 81-97, wherein gedatolisib, or a pharmaceutically acceptable salt, solvate, or ester thereof, is administered at a dosage of 240 mg weekly.
   104. Gedatolisib, or a pharmaceutically acceptable salt, solvate, or ester thereof, for use in a method of treating a hormonally driven disorder other than breast cancer in a human subject, the method comprising:
administering to a human subject with a hormonally driven disorder other than breast cancer:
gedatolisib, or a pharmaceutically acceptable salt, solvate, or ester thereof, at a dosage of 100-300 mg once a week for an administration period of three weeks;
discontinuing administration of gedatolisib, or pharmaceutically acceptable salt, solvate, or ester thereof, for a discontinuation period of one week; and
resuming administration of gedatolisib, or pharmaceutically acceptable salt, solvate, or ester thereof, once a week following the discontinuation period,
wherein the administration period of three weeks and the discontinuation period of one week constitutes a cycle, wherein the cycle is repeated for at least two cycles; and
a hormone therapy that is administered for the at least two cycles.
   105. Gedatolisib, or a pharmaceutically acceptable salt, solvate, or ester thereof, for use according to embodiment 104, wherein the hormonally driven disorder is prostate cancer and the hormone therapy is darolutamide.
   106. Gedatolisib, or a pharmaceutically acceptable salt, solvate, or ester thereof, for use according to embodiment 105, wherein darolutamide is administered at a dosage of 600 mg twice daily.
   107. Gedatolisib, or a pharmaceutically acceptable salt, solvate, or ester thereof, for use according to embodiment 105, wherein the gedatolib dosage is 120 mg/week or 180 mg/week.
   108. Gedatolisib, or a pharmaceutically acceptable salt, solvate, or ester thereof, for use in a method of treating prostate cancer in a human subject, the method comprising:
administering to a human subject with prostate cancer:
(c) gedatolisib, or a pharmaceutically acceptable salt, solvate, or ester thereof, at a dosage of 100-300 mg once a week for an administration period of three weeks;
   discontinuing administration of gedatolisib, or pharmaceutically acceptable salt, solvate, or ester thereof, for a discontinuation period of one week; and
resuming administration of gedatolisib, or pharmaceutically acceptable salt, solvate, or ester thereof, once a week following the discontinuation period; and
(d) darolutamide at a dosage of 600 mg twice daily for an administration period of three weeks;
   discontinuing administration of darolutamide for a discontinuation period of one week; and
resuming administration of darolutamide at a dosage of 600 mg twice daily following the discontinuation period;
wherein the administration period of three weeks and the discontinuation period of one week constitutes a cycle, wherein the cycle is repeated for at least two cycles; and
wherein the discontinuation period for administering gedatolisib, or a pharmaceutically acceptable salt, solvate, or ester thereof, and the discontinuation period for administering darolutamide are concurrent.
   109. Gedatolisib, or a pharmaceutically acceptable salt, solvate, or ester thereof, for use in a method of treating an endometrial disorder in a human subject, the method comprising:
administering to a human subject with an endometrial disorder:
(d) gedatolisib, or a pharmaceutically acceptable salt, solvate, or ester thereof, at a dosage of 100-300 mg once a week for an administration period of three weeks;
   discontinuing administration of gedatolisib, or pharmaceutically acceptable salt, solvate, or ester thereof, for a discontinuation period of one week; and
resuming administration of gedatolisib, or pharmaceutically acceptable salt, solvate, or ester thereof, once a week following the discontinuation period;
(e) a hormone therapy; and
(f) a CDK4/6 inhibitor for an administration period of three weeks;
   discontinuing administration of the CDK4/6 inhibitor for a discontinuation period of one week; and
resuming administration of the CDK4/6 inhibitor following the discontinuation period;
wherein the administration period of three weeks and the discontinuation period of one week constitutes a cycle, wherein the cycle is repeated for at least two cycles;
wherein the discontinuation period for administering gedatolisib, or a pharmaceutically acceptable salt, solvate, or ester thereof, and the discontinuation period for administering the CDK 4/6 inhibitor are concurrent; and
wherein the endometrial disorder is selected from the group consisting of endometriosis, endometrial hyperplasia, endometrial carcinoma, endometrial intraepithelial neoplasia (EIN), intrauterine adhesions (IUA), adenomyosis, endometritis, endometrial and/or uterine polyps and Asherman syndrome.

The present invention is further illustrated by the following examples, which should not be construed as further limiting. The contents of FIGURES and all references, patents and published patent applications cited throughout this application are expressly incorporated herein by reference.

### EXAMPLES

### Example 1: a Phase 1/2 Study of Gedatolisib in Combination with Darolutamide in Metastatic Castration-Resistant Prostate Cancer (mCRPC)

This example describes a Phase 1/2 study of gedatolisib in combination with the non-steroidal ARsi darolutamide in metastatic castration-resistant prostate cancer (mCRPC). Gedatolisib was tested at two starting doses: 120 mg and 180 mg i.v. once per week, according to a schedule of three weeks on/one week off. Darolutamide was used at the recommended dose of 600 mg administered twice daily orally, for a total dosage of 1200 mg/day. The results for the combined arms showed that the six-month radiographic progression free survival (rPFS) was 66%. Moreover, no treatment-related discontinuations or dosage reductions were needed and the rate of adverse events was low (e.g., less than 3% of patients experienced grade 3 stomatitis).

### Study Rationale

For patients with newly diagnosed mCRPC lacking a BRCA mutation, second-generation AR inhibitors, abiraterone or enzalutamide, and the chemotherapy, docetaxel, are the current standard of care first-line treatments. For patients who received docetaxel as first-line therapy, abiraterone and enzalutamide are the preferred second-line treatment options. For patients who received an ARi as first-line therapy, docetaxel is the preferred option but treatment with a different ARi than the patient received initially is also a recommended option. However, the median progression-free survival reported in randomized clinical studies for these second-line regimens is only 5-7 months, highlighting the need for more effective treatment options.

Prostate cancer resistance to anti-androgen therapies can arise from autocrine ligand production, intramural androgen biosynthesis, AR gene amplification, somatic mutation in the ligand binding domain (LBD) of AR and altered splicing of AR messenger ribonucleic acid (mRNA) resulting in loss of the LBD, the so-called AR variant (AR-v7) form. Lack of response of men with mCRPC to abiraterone and enzalutamide was associated with detection of AR-v7 mRNA in circulating tumor cells (CTCs) using an RNA-based polymerase chain reaction (PCR) assay (Antonarakis et al. (2014) N Engl J Med. 371:1028-1038).

The oncogenic PI3K/AKT/mTOR pathway is activated in the majority of advanced CRPC (Crumbaker et al. (2017) Cancers (Basel) 9:34). The PI3K and AR pathways cross-regulate each other through reciprocal negative feedback, and ARi drug resistance can be induced through the activation of PI3K/mTOR signaling. Combined inhibition of these pathways was shown to cause regression in prostate cancer xenograft models (Carver et al. (2011) Cancer Cell 19:575-586; Mulholland et al. (2011) Cancer Cell. 19:792-804). In the clinic, the upregulation of the PI3K/AKT/mTOR pathway is associated with disease progression, therapeutic resistance, and poor outcomes in prostate cancer (Shorning et al. (2020) Int J Mol Sci. 21:4507).

Numerous preclinical studies have demonstrated interaction between the AR and PI3K-mTOR pathways through reciprocal negative feedback, whereby inhibition of one pathway cross activates the other (Carver et al. (2011) Cancer Cell 19:575-586; Mulholland et al. (2011) Cancer Cell. 19:792-804), suggesting that combining a PI3K-mTOR inhibitor with an ARi may induce a synergistic antitumor effect in nmCRPC, mHSPC, or mCRPC patients, including those who had progressed on prior treatment with an ARi. Thus, there is a clear unmet need for nmCRPC, mHSPC, or mCRPC for agents that specifically target this pathway.

Nonclinical evidence supporting this hypothesis was obtained when the prostate C4-2 (castrated) cell line mouse xenograft model was evaluated with the dual PI3K/mTOR inhibitor, gedatolisib, and the ARi, enzalutamide, alone and in combination. In that study, tumor growth inhibition (TGI) was 86% for gedatolisib as a single agent and 84% for enzalutamide alone. When gedatolisib was combined with enzalutamide, TGI was 116%.

### Hormone Therapy

The study is designed to evaluate gedatolisib in combination with the hormone therapy darolutamide. Darolutamide (NUBEQA® USPI) is a potent and selective nonsteroidal AR antagonist that is approved for the treatment of nmCRPC and mHSPC in combination with docetaxel. Darolutamide is comprised of a mixture of 2 diastereomers, (S,R)-darolutamide (ORM-16497) and (S,S)-darolutamide (ORM-16555), which interconvert via the major metabolite keto-darolutamide (ORM-15341) preferentially to (S,S)-darolutamide; all three compounds show similar pharmacologic activity (Moilanen et al. (2015) Sci Rep. 5:12007; Taavitsainen et al. (2021)

### Drug Metab Dispos. 49:420-433).

In preclinical studies, darolutamide demonstrated higher binding affinity compared with other AR antagonists (such as enzalutamide and apalutamide [ARN-509]), an antiproliferative effect and tumor growth inhibition in AR-overexpressing cells, and activity against AR mutants linked to drug resistance. In addition, darolutamide is different from other nonsteroidal AR antagonists with respect to its negligible blood-brain barrier penetration (Moilanen et al. (2015) Sci Rep. 5:12007). In early-phase clinical trials with mCRPC patients, darolutamide has shown a good safety profile and significant reductions in PSA levels (Fizazi et al. (2014) Lancet Oncol. 15:975-985; Massard et al. (2016) Eur Urol. 69:834-840; Matsubara et al. (2017) Cancer Chemother Pharmacol. 80:1063-1072; Shore et al. (2018) Eur Urol Focus 4:547-553).

The BOIN utility score design was chosen to select the RP2D at the optimal biological dose (OBD) level, utilizing both safety and efficacy assessments (Zhou et al. (2019) Stat Med. 38:5299-5316).

### Gedatolisib Dose Selection Rationale

Two starting doses of gedatolisib, 120 mg and 180 mg, administered via IV once weekly for 3 weeks on/1 week off, were selected based on available data from prior studies. Gedatolisib was evaluated at dose levels between 10 mg to 319 mg administered IV weekly in a first-in-human study in subjects with solid tumors. In subsequent studies in advanced breast cancer, a 180 mg dose was administered either once weekly or a schedule of once weekly for 3 weeks on/1 week off. Based on the results from these studies, gedatolisib 180 mg administered once weekly for 3 weeks on/1 week off was selected as the dose for an ongoing Phase 3 study in subjects with advanced breast cancer.

Based on available PK data from prior studies, gedatolisib dose levels of 120 mg and 180 mg, administered using a 3 weeks on/1 week off schedule, were chosen. These doses provide clear separation of PK parameters between the dosing arms and will enable assessment of the optimal biological dose level.

### Effect of Darolutamide on Gedatolisib Pharmacokinetics

Darolutamide, an inhibitor of OATPIB1 and BCRP, has been shown to increase the plasma exposure of rosuvastatin, a substrate of OATPIB1 and BCRP, by approximately 5-fold in a clinical drug-drug interaction (DDI) study. In this study, rosuvastatin bioavailability may have been reduced due to administration with food, thereby magnifying the DDI effect of BCRP and OATPIB1 inhibition (Zurth et al. (2019) Eur J Drug Metab Pharmacokinet. 44:747-759). Therefore, the results of this DDI study are considered to be a worst-case scenario arising from inhibition of two transporters and modified rosuvastatin bioavailability due to food effect; the contribution of individual components of drug interaction was not elucidated in this study.

Based on the following considerations, the effect of darolutamide on gedatolisib plasma exposure is not expected to be significant.
(i) Gedatolisib is a substrate for BCRP and is not a substrate for OATPIB1;
(ii) Unlike rosuvastatin that was administered orally in the above DDI study, gedatolisib is administered intravenously. Therefore, inhibition of intestinal BCRP (that resulted in higher fraction of rosuvastatin absorbed) is not relevant with respect to gedatolisib pharmacokinetics. Inhibition of BCRP in the liver by darolutamide has the potential to modulate gedatolisib pharmacokinetics. However, it should be noted that the International Transporter Consortium has recently concluded that the risk of clinical DDI upon BCRP inhibition in the liver is low (Taskar et al. (2022) Clin Pharmacol Ther. 112:573-592);
(iii) Since gedatolisib is administered intravenously once weekly in a three-weeks-on/one-week-off schedule, accumulation due to DDI, if any, should be minimal.

Although the effect of darolutamide on gedatolisib pharmacokinetics is not expected to be significant, multiple doses of gedatolisib (120 mg, Arm 1; 180 mg, Arm 2) will be evaluated in this study. Plasma samples will be collected for characterization of gedatolisib pharmacokinetics to assess the magnitude of drug interaction. This will inform dose selection for Phase 2 Dose Expansion portion of the study.

Previous gedatolisib dose escalation studies included doses as high as 319 mg once weekly. Based on the safety profile from the totality of clinical data, the selected gedatolisib doses for this study (in combination with darolutamide) are 120 mg and 180 mg IV weekly for 3 weeks on (Days 1, 8, and 15) followed by 1 week off.

Combined P-gp and strong or moderate CYP3A4 inhibitors/inducers have the potential to increase/decrease darolutamide plasma concentrations and these agents are recommended to be avoided according to the darolutamide package insert. Available in vitro and clinical PK data indicate that gedatolisib is not a CYP3A4 inhibitor or inducer and gedatolisib is not expected to have an effect on darolutamide pharmacokinetics. Therefore, subjects enrolled in this study will receive darolutamide at the recommended dose of 600 mg administered twice daily.

### Study Design

Described below is a Phase 1/1b/2, open label, randomized, dose finding and dose expansion study to evaluate the safety, preliminary efficacy, and PK of gedatolisib in combination with darolutamide in subjects with mCRPC.

### Phase 1: Optimal Dose Finding Study

### Randomized BOIN Design (Arms 1 and 2; Completed)

There were 2 different dose arms of gedatolisib (administered once weekly for 3-weeks-on/1-week-off) in combination with darolutamide 600 mg BID that were evaluated: Arm 1 (120 mg) and Arm 2 (180 mg). A total of 38 subjects were enrolled, with 19 subjects in each arm. A modified BOIN dose de-escalation rule with a target DLT rate of ≤27% was used for safety assessment; however, DLTs were not observed and dose de-escalation was not needed.

Intermittent dosing of both 120 mg and 180 mg gedatolisib was reasonably tolerated. All subjects were followed for assessment of potential late onset toxicities for no less than 90 days after the first dose. Arm 1 (120 mg) and Arm 2 (180 mg) continued to enroll until both arms reached 19 treated subjects per arm (Table 2). Both arms cleared the safety and futility stopping rules. The risk-benefit trade-off using the BOIN utility score and the totality of the safety data, including follow up for late onset toxicities were evaluated.

### Results

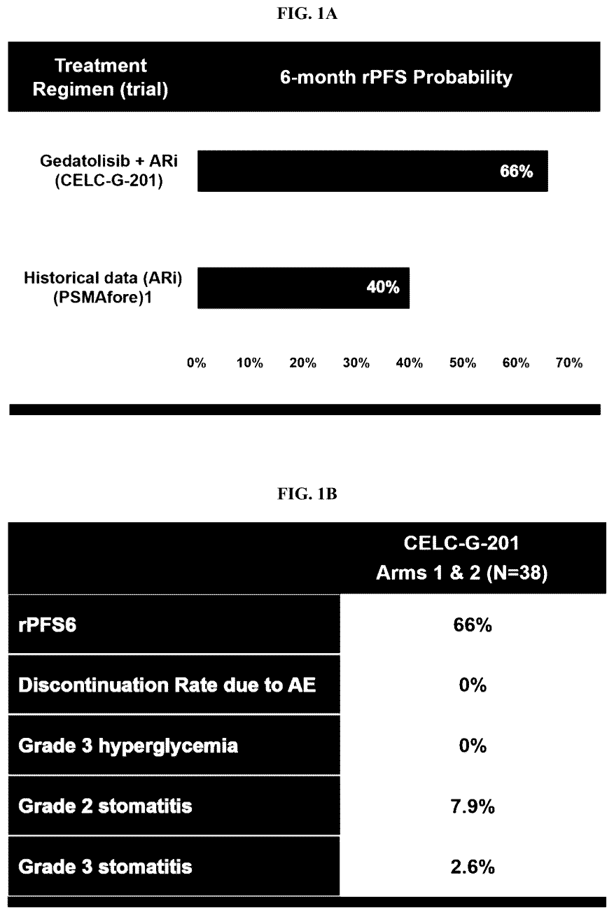

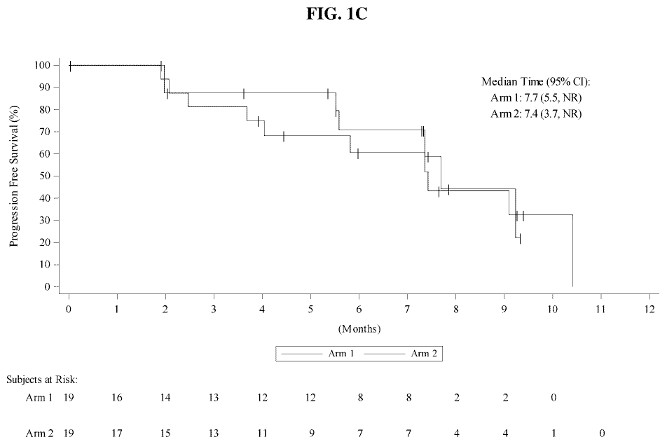

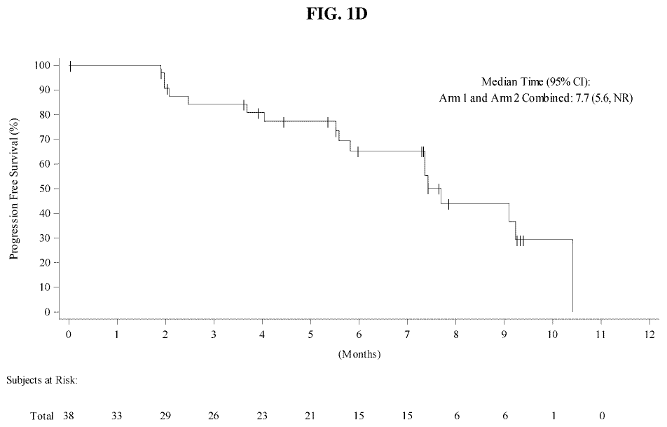

In the Phase 1 portion of the clinical trial evaluating gedatolisib plus darolutamide in men with metastatic castration resistant prostate cancer (“mCRPC”), the six-month radiographic progression free survival (“rPFS”) rate was 66%. FIG. 1A shows a graphic of the 6-month rPFS probability for the gedatolisib and ARi treatment arm as compared to ARi alone (darolutamide). As can be observed, the gedatolisib and Ari treatment arm compares favorably to historical data for ARi monotherapy.

FIG. 1C shows a Kaplan Meier curve of radiological progression free survival (rPFS) for both treatment arms of the phase I study. FIG. 1D is a Kaplan Meier curve for combined rPFS data for both arm 1 and arm 2 of the phase I study.

A summary of rPFS for each arm of the study as well as the combined data is provided in Table 1 below.

TABLE 1
Radiological Progression-Free Survival.

| | Arm 1 | Arm 2 | Overall |
|---|---|---|---|
| | (N = 19) | (N = 19) | (N = 38) |
| Probability of being event free at Month 6 | 70.7 (51.1, | 60.6 (42.5, | 65.3 (52.4, |
| (80% CI) [1] | 83.6) | 74.6) | 75.5) |
| Probability of being event free at Month 6 | 70.7 (38.6, | 60.6 (32.1, | 65.3 (44.7, |
| (95% CI) [2] | 88.1) | 80.2) | 79.8) |
| Median PFS (months) (1Q, 3Q) [3] | 7.7 (5.6, | 7.4 (3.9, | 7.7 (5.5, |
| | 9.2) | 10.4) | 10.4) |
| Median PFS follow-up (months) (1Q, 3Q) | 7.3 (3.6, | 7.7 (4.4, | 7.4 (3.9, |
| [4] | 7.9) | 9.4) | 9.3) |

[1], [2], [3] Calculated from the Kaplan-Meier Estimates using Greenwood.
[4] Calculated from the reverse Kaplan-Meier method

No patients discontinued treatment due to a treatment-related adverse effects and no dose reductions were required with gedatolisib or darolutamide. Indeed, FIG. 1B shows discontinuation rate due to adverse events (0%), thereby establishing that the gedatolisib and ARi treatment is well tolerated in treated subjects. The data shows that no Grade 3 hyperglycemia was reported. Grade 2-3 stomatitis was reported in only four (10.5%) patients: three (7.9%) Grade 2 and one (2.6%) Grade 3.

### EQUIVALENTS

Those skilled in the art will recognize, or be able to ascertain using no more than routine experimentation, many equivalents of the specific embodiments of the invention described herein. Such equivalents are intended to be encompassed by the following claims:

1. A method of treating a hormonally driven disorder in a human subject, the method comprising administering to a human subject with a hormonally driven disorder:
gedatolisib, or a pharmaceutically acceptable salt, solvate, or ester thereof, at a dosage of 100-300 mg weekly for at least two weeks; and
a hormone therapy for at least two weeks;
wherein the hormonally driven disorder is not breast cancer.

2. The method of claim 1, wherein the hormonally driven disorder is prostate cancer.

3. The method of claim 2, wherein the prostate cancer has become hormone resistant at time of administering gedatolisib, or a pharmaceutically acceptable salt, solvate, or ester thereof.

4. The method of claim 2, wherein the hormone therapy comprises administering to the subject an androgen receptor signaling inhibitor (ARsi) selected from the group consisting of darolutamide, apalutamide, bicalutamide, enzalutamide, flutamide, nilutamide and topilutamide.

5. (canceled)

6. The method of claim 4, wherein the ARsi is darolutamide.

7. The method of claim 6, wherein darolutamide is orally administered at a dosage of 600 mg twice daily.

8. The method of claim 2, wherein the hormone therapy comprises administering to the subject a steroidal anti-androgen, an androgen synthesis inhibitor, an androgen receptor pathway inhibitor, an androgen receptor protein degrader, or an N-terminal domain (NTD) androgen receptor inhibitor.

9.-19. (canceled)

20. The method of claim 1, wherein gedatolisib, or a pharmaceutically acceptable salt, solvate, or ester thereof, is administered at a dosage of 100 mg, 120 mg, 150 mg, 180 mg, 210 mg, or 240 mg weekly.

21.-25. (canceled)

26. The method of claim 1, wherein gedatolisib, or a pharmaceutically acceptable salt, solvate, or ester thereof, and the hormone therapy are administered for at least four weeks.

27. The method of claim 1, which further comprises treating the human subject with one or more additional therapies effective in the hormonally driven disorder.

28. The method of claim 27, wherein the one or more additional therapies comprise administering to the subject one or more chemotherapeutic drugs or therapies that target oncogenic proteins.

29. The method of claim 27, wherein the one or more additional therapies comprise administering to the subject one or more immune checkpoint inhibitors.

30. (canceled)

31. The method of claim 27, wherein the one or more additional therapies comprise administering to the subject one or more receptor tyrosine kinase (RTK) inhibitors.

32. The method of claim 27, wherein the one or more additional therapies comprise administering to the subject one or more CDK 4/6 inhibitors.

33. The method of claim 27, wherein the one or more additional therapies comprise administering to the subject one or more epigenetic-targeted therapies.

34. (canceled)

35. A method of treating prostate cancer in a human subject, the method comprising administering to a human subject with prostate cancer:
gedatolisib, or a pharmaceutically acceptable salt, solvate, or ester thereof, at a dosage of 100-300 mg weekly for at least two weeks; and
a hormone therapy for at least two weeks.

36. The method of claim 35, wherein the prostate cancer is hormone driven at time of administering gedatolisib, or a pharmaceutically acceptable salt, solvate, or ester thereof.

37. (canceled)

38. The method of claim 35, wherein the hormone therapy comprises administering to the subject an androgen receptor signaling inhibitor (ARsi).

39. The method of claim 38, wherein the ARsi is a non-steroidal androgen receptor inhibitor is selected from the group consisting of darolutamide, apalutamide, bicalutamide, enzalutamide, flutamide, nilutamide and topilutamide.

40. The method of claim 39, wherein the ARsi is darolutamide.

41. The method of claim 40, wherein darolutamide is orally administered at a dosage of 600 mg twice daily.

42. The method of claim 35, wherein the hormone therapy comprises administering to the subject a steroidal anti-androgen, an androgen synthesis inhibitor, an androgen receptor pathway inhibitor, an androgen receptor protein degrader, or an N-terminal domain (NTD) androgen receptor inhibitor.

43.-49. (canceled)

50. The method of claim 35, wherein gedatolisib, or a pharmaceutically acceptable salt, solvate, or ester thereof, is administered at a dosage of 100 mg, 120 mg, 150 mg, 180 mg, 210 mg, or 240 mg weekly.

51.-55. (canceled)

56. The method of claim 35, wherein gedatolisib, or a pharmaceutically acceptable salt, solvate, or ester thereof, and the hormone therapy are administered for at least four weeks.

57. The method of claim 35, which further comprises treating the human subject with one or more additional therapies effective in prostate cancer.

58.-64. (canceled)

65. A method of treating prostate cancer in a human subject, the method comprising administering to a human subject with prostate cancer:
gedatolisib, or a pharmaceutically acceptable salt, solvate, or ester thereof, at a dosage of 100-300 mg weekly; and
darolutamide at a dosage of 600 mg twice daily.

66.-80. (canceled)

81. A method of treating an endometrial disorder in a human subject, the method comprising administering to a human subject with an endometrial disorder:
gedatolisib, or a pharmaceutically acceptable salt, solvate, or ester thereof, at a dosage of 100-300 mg weekly for at least two weeks; and
a hormone therapy for at least two weeks;
wherein the endometrial disorder is selected from the group consisting of endometriosis, endometrial hyperplasia, endometrial carcinoma, endometrial intraepithelial neoplasia (EIN), intrauterine adhesions (IUA), adenomyosis, endometritis, endometrial and/or uterine polyps and Asherman syndrome.

82.-103. (canceled)

104. A method of treating a hormonally driven disorder other than breast cancer in a human subject comprising:
administering to a human subject with a hormonally driven disorder other than breast cancer:
gedatolisib, or a pharmaceutically acceptable salt, solvate, or ester thereof, at a dosage of 100-300 mg once a week for an administration period of three weeks;
discontinuing administration of gedatolisib, or pharmaceutically acceptable salt, solvate, or ester thereof, for a discontinuation period of one week; and
resuming administration of gedatolisib, or pharmaceutically acceptable salt, solvate, or ester thereof, once a week following the discontinuation period,
wherein the administration period of three weeks and the discontinuation period of one week constitutes a cycle, wherein the cycle is repeated for at least two cycles; and
a hormone therapy that is administered for the at least two cycles.

105.-107. (canceled)

108. A method of treating prostate cancer in a human subject comprising:
administering to a human subject with prostate cancer:
(a) gedatolisib, or a pharmaceutically acceptable salt, solvate, or ester thereof, at a dosage of 100-300 mg once a week for an administration period of three weeks;
discontinuing administration of gedatolisib, or pharmaceutically acceptable salt, solvate, or ester thereof, for a discontinuation period of one week; and
resuming administration of gedatolisib, or pharmaceutically acceptable salt, solvate, or ester thereof, once a week following the discontinuation period; and
(b) darolutamide at a dosage of 600 mg twice daily for an administration period of three weeks;
discontinuing administration of darolutamide for a discontinuation period of one week; and
resuming administration of darolutamide at a dosage of 600 mg twice daily following the discontinuation period;
wherein the administration period of three weeks and the discontinuation period of one week constitutes a cycle, wherein the cycle is repeated for at least two cycles; and
wherein the discontinuation period for administering gedatolisib, or a pharmaceutically acceptable salt, solvate, or ester thereof, and the discontinuation period for administering darolutamide are concurrent.

109. A method of treating an endometrial disorder in a human subject comprising:
administering to a human subject with an endometrial disorder:
(a) gedatolisib, or a pharmaceutically acceptable salt, solvate, or ester thereof, at a dosage of 100-300 mg once a week for an administration period of three weeks;
discontinuing administration of gedatolisib, or pharmaceutically acceptable salt, solvate, or ester thereof, for a discontinuation period of one week; and
resuming administration of gedatolisib, or pharmaceutically acceptable salt, solvate, or ester thereof, once a week following the discontinuation period;
(b) a hormone therapy; and
(c) a CDK4/6 inhibitor for an administration period of three weeks;
discontinuing administration of the CDK4/6 inhibitor for a discontinuation period of one week; and
resuming administration of the CDK4/6 inhibitor following the discontinuation period;
wherein the administration period of three weeks and the discontinuation period of one week constitutes a cycle, wherein the cycle is repeated for at least two cycles;
wherein the discontinuation period for administering gedatolisib, or a pharmaceutically acceptable salt, solvate, or ester thereof, and the discontinuation period for administering the CDK 4/6 inhibitor are concurrent; and
wherein the endometrial disorder is selected from the group consisting of endometriosis, endometrial hyperplasia, endometrial carcinoma, endometrial intraepithelial neoplasia (EIN), intrauterine adhesions (IUA), adenomyosis, endometritis, endometrial and/or uterine polyps and Asherman syndrome.
